(19) United States
(12) Patent Application Publication
(10) Pub. No.: US 2022/0259646 A1
(43) Pub. Date: Aug. 18, 2022

(54) COMPOSITIONS AND METHODS OF LABELING NUCLEIC ACIDS AND SEQUENCING AND ANALYSIS THEREOF

(71) Applicant: King Abdullah University of Science and Technology, Thuwal (SA)

(72) Inventors: Mo Li, Thuwal (SA); Chongwei Bi, Thuwal (SA); Lin Wang, Thuwal (SA)

(21) Appl. No.: 17/436,496

(22) PCT Filed: Mar. 4, 2020
(86) PCT No.: PCT/IB2020/051894

§ 371 Date: Sep. 3, 2021

Related U.S. Application Data
(60) Provisional application No. 62/813,605, filed on Mar. 4, 2019.
(60) Provisional application No. 62/899,142, filed on Sep. 11, 2019.
(60) Provisional application No. 62/899,432, filed on Sep. 12, 2019.

(51) Int. Cl. C12Q 1/6869; C12Q 1/6883

(52) U.S. Cl. CPC C12Q 1/6869; G16B 20/20; C12Q 1/6883

(57) ABSTRACT

Compositions and methods labeling individual nucleic acid (e.g., DNA) molecules with a unique molecular identifier (UMI), followed by amplification by PCR are provided. The PCR amplicons can be grouped by the UMI they contain and traced back to the original molecule. More specifically, the grouped reads with the same UMI represent one original nucleic acid (e.g., DNA) molecule, meaning they share the same nucleic acid sequence. Methods of sequencing the labeled nucleic acid are also provided. The methods can include determination of a consensus sequence, which thus eliminates errors that may be introduced in the amplification and sequencing process. Such methods can be used in, for example, the detection of rare genetic variants.

### CROSS-REFERENCE TO RELATED APPLICATION

This application claims priority to and benefit of U.S. Ser. No. 62/813,605, filed Mar. 4, 2019, U.S. Ser. No. 62/899,142, filed Sep. 11, 2019, and U.S. Ser. No. 62/899,432, filed Sep. 12, 2019, each of which are specifically incorporated by reference herein in their entireties.

### FIELD OF THE INVENTION

The field of the invention generally relates to compositions and methods for labeling and optionally amplifying a nucleic acid sequence typically for sequencing.

### BACKGROUND OF THE INVENTION

Life came from the same ancestor billion years ago. During the long evolutionary time, variations took place, and its accumulation leads to a diverse lifespan in the world. Mouse ages and dies in less than 3.5 years, while its African cousin Heterocephalus glaber, known as the naked mole rat, exists a maximum lifespan of more than 30 years (Kim et al., Nature 479, 223-227, doi:10.1038/nature10533 (2011)). Given the truth that mice and naked mole rats show a comparative body size, it challenges the long-standing assumption that an animal with a higher body mass will have greater longevity (Prothero & Jurgens, Basic Life Sci 42, 49-74 (1987)). Over the past decade, the study of the biological basis of aging has provided evidence that the time-dependent accumulation of lesions in cells contributes significantly to aging (Lopez-Otin et al., Cell 153, 1194-1217, doi:10.1016/j.cell.2013.05.039 (2013), Gems & Partridge, Annu Rev Physiol 75, 621-644, doi:10.1146/annurev-physiol-030212 183712 (2013), Kirkwood, Cell 120, 437-447, doi:10.1016/j.cell.2005.01.027 (2005)). In particular, the alteration of mitochondrial and nuclear genome has emerged as driving instigators for the progressive deterioration of cell vitality in stem cells and mouse model during aging (Behrens et al., Nat Cell Biol 16, 201-207, doi:10.1038/ncb2928 (2014), Kauppila et al., Cell Metab 25, 57-71, doi:10.1016/j.cmet.2016.09.017 (2017), Trifunovic et al., Nature 429, 417-423, doi:10.1038/nature02517 (2004)). Nonetheless, variations are random events and tend to be unique in a single cell or a small portion of a bulk of cells, hindering current population-based genomic study and stalling understanding of the causality of the genomic alteration and aging (Vijg & Montagna, Translational Medicine of Aging 1, 5-11, doi:10.1016/j.tma.2017.09.003 (2017)).

### Mitochondrial Introduction and its Function in Cell

Mitochondria are semiautonomous organelles that exist in most eukaryotic cells. Over its long evolution, mitochondrion has dedicated itself to be a “powerhouse” for the cell and surrendered most of its genomic material to the nucleus. The circular mitochondrial genome (mtDNA) in modern day humans is about 16 kb, tightly packaged as a nucleoid within the mitochondrial matrix. It consists of 37 genes encoding two mitochondrial ribosome-coding RNAs, 22 transfer RNAs and 13 vital constituents of the oxidative phosphorylation (OXPHOS) complexes, which embed in the mitochondrial inner membrane (Taanman, Biochim Biophys Acta 1410, 103-123 (1999)). In addition to producing the majority of cellular ATP, increasing evidence showed mitochondria are also key components in cellular metabolic and signaling processes such as beta-oxidation of fatty acid, iron-sulfur cluster synthesis, calcium signaling, and apoptosis (van der Giezen & Tovar, EMBO Rep 6, 525-530, doi:10.1038/sj.embor.7400440 (2005)). The multi-function feature of mitochondria makes it no longer a simple “energy factory,” but a hub for regulating the growth and development of the cell. Therefore, faithful and effective mitochondrial function is indispensable for cell survival and biotic health.

Mitochondrial organization is a conserved feature. For example. mtDNA in a human fibroblast is packaged within nucleoids distributed within tubular mitochondria around the nucleus (Friedman & Nunnari, Nature 505, 335-343, doi:10.1038/nature12985 (2014)). A similar distribution is seen in a yeast cell with nucleoids within mitochondria.

### Mitochondrial Genome Mutation

In mammals, different cell types contain distinct copy number of mitochondria, and single mitochondrion can contain several copies of mtDNA. This results in a variety of number of mtDNA per cell in the human body, ranging from as much as ˜77,000 in oocytes to as few as 171 in the lung cells (Duran et al., ASRM 92, 5218 (2009), D'Erchia et al., Mitochondrion 20, 13-21, doi:10.1016/j.mito.2014.10.005 (2015)). The mitochondrial genome is maintained (replication and repair) by DNA polymerase γ (pol γ). This polymerase is encoded by a nuclear gene termed POLG and it is the sole one of 16 cellular DNA polymerases known to function in mitochondria in human (Bebenek & Kunkel, Adv Protein Chem 69, 137-165,doi:10.1016/S0065-3233(04)69005-X (2004)). Mitochondria have a frequent DNA replication to maintain its function in cells. Errors introduced during the replication and the subsequently inefficient DNA repair lead to a significantly higher mutation rate (about 24-fold in human) in the mtDNA than the nuclear DNA (Kauppila et al., Cell Metab 25, 57-71, doi:10.1016/j.cmet.2016.09.017 (2017), Lynch & Schaack, Science 311, 1727-1730, doi:10.1126/science.1118884 (2006)). Inherent mitochondrial mutations have been found to cause a series of metabolic diseases, such as

Leber's hereditary optic neuropathy (LHON), Mitochondrial encephalomyopathy, lactic acidosis and stroke-like episodes (MELAS) and myoclonus epilepsy and ragged-red fibers (MERRF) (Schon et al., Nat Rev Genet 13, 878-890, doi:10.1038/nrg3275 (2012)). Furthermore, the mutation rate of mtDNA increase with age both in mouse models and in humans (Cortopassi & Arnheim, Nucleic Acids Res 18, 6927-6933 (1990), Pikó et al., Mech Ageing Dev 43, 279-293 (1988)).

To date, both single nucleotide mutations and structural rearrangements have been identified in mitochondria. An increasing number of studies have proved that the integrity of the mitochondrial genome has a crucial impact on human reproductive health, disease, and aging (Stewart & Chinnery, Nat Rev Genet 16, 530-542, doi:10.1038/nrg3966 (2015)). In consequence, it is important to have a better understanding of different aspects of mtDNA mutations and characterize their correlation to human disease and aging.

### MtDNA Mutation and Aging

Aging is a progressive decline of organismal function with time, and it commonly exists in most organisms on the planet. Mitochondrial dysfunction has been linked with aging. Early studies speculated that aging stems from the accumulation of reactive oxygen species (ROS) introduced by mitochondrial mutation and the alteration of mtDNA contribute to the aging process (Harman, J Am Geriatr Soc 20, 145-147 (1972), Linnane et al., Mutat Res 275, 195-208 (1992)). However, there is still no clear conclusion regarding the causality of mitochondria and aging. The controversy mainly comes from the suspicion in whether the mitochondrial mutation rate is significant enough to cause a global aging process (Khrapko & Vijg, Trends Genet 25, 91-98, doi:10.1016/j.tig.2008.11.007 (2009)).

Evidence that mitochondrial dysfunction contributes to aging comes from the POLG mutation mouse. The PolgAD257A mutation in the mouse genome leads to a disability of the proofreading function of mitochondrial DNA polymerase while remaining its polymerase function. This results in a threefold to fivefold higher mutation rate in mutator mouse than wild-type, and eventually gives rise to an accumulation of mutated mtDNA with time. Mouse harbor this homozygous mutation showed an accelerated aging symptom, including reduced lifespan, weight loss, hair loss, osteoporosis, and low fertility (Trifunovic et al., Nature 429, 417-423, doi:10.1038/nature02517 (2004)). In addition, the accelerated mtDNA mutations by PolgAD257A change cause a blockage during hematopoietic stem cells (HSCs) differentiation (Norddahl et al., Cell Stem Cell 8, 499-510, doi:10.1016/j.stem.2011.03.009 (2011)). These data show a strong notion that the accumulated mutations of mtDNA bring out a stem cell dysfunction and potentially contribute to aging.

### MtDNA Heteroplasmy and its Problem for Sequencing

Since there are hundreds to thousands of mitochondria per cell and they form a connected network to function, mutation in one mtDNA may not have a phenotype. Indeed, the polyploid property of mitochondria often results in the coexistence of wild-type and mutational mtDNA in the single cell or a population (Chinnery & Hudson, Br Med Bull 106, 135-159, doi:10.1093/bmb/1dt017 (2013)). These mtDNA mutations exhibit a highly uneven distribution over different tissues, cell types, and even individual cells. This is partially due to the rate of mtDNA replication varies in cells and is regardless of the cell division (Friedman & Nunnari, Nature 505, 335-343, doi:10.1038/nature12985 (2014)).

It is widely believed that the mutational burden of cellular mitochondria has to surpass a threshold to reveal a phenotype, which is a phenomenon termed heteroplasmy. More specifically, healthy cells can exist as a small proportion of mutated mtDNA. When the proportion exceeds a threshold, disease-related phenotype will show up. This threshold varies for different mutations and cell types. For example, an 80-90% mutation is generally needed for point mutation related mitochondrial disease (White et al., Am J Hum Genet 65, 474-482, doi:10.1086/302488 (1999)).

The heteroplasmy nature of mtDNA makes it challenging to study the mitochondrial genome using the current population-based next-generation sequencing (NGS) method. The prevalent strategy is to do a PCR-based ultra-deep next generation sequencing (NGS) with coverage more than 2000X. Although this kind of work indicates that at least one of two hundreds of healthy human harbor a common mutation in the mitochondrial genome (Elliott et al., Am J Hum Genet 83, 254-260, doi:10.1016/j.ajhg.2008.07.004 (2008)), the sensitivity of the-state-of-the-art method is still limited and unable to satisfy the analysis of mtDNA mutation in different contexts. Illumina NGS has an intrinsic sequencing error of −0.2%.

The current mtDNA enrichment is achieved by regular PCR to several fragments, which could introduce unintended amplification of nuclear mitochondrial DNA sequences (NUMTs) (Payne et al., Methods Mol Biol 1264, 59-66, doi:10.1007/978-1-4939-2257-4_6 (2015)). In addition, the amplification and PCR-based library preparation steps will introduce a nonnegligible amount of errors Amplification by PCR introduces errors and biases due to the property of the DNA polymerase and the technique itself. These errors combined with the 0.1-1% of typical intrinsic sequencing error will make it even harder to find rare mutations, especially in a complex genetic background like human genome.

Further complicating matters, the disease-related mitochondrial mutation load is usually very low at tissue level but high in individual cells. A population-based analysis of mitochondrial mutation is inefficient in this circumstance. Taken together, a new strategy is needed to overcome these hurdles.

Furthermore, NGS by Illumina platform generates relatively short-reads, which are not suitable for detecting and haplotyping the rare mutations and calling structural variants (Lou et al., Proc Natl Acad Sci U S A 110, 19872-19877, doi:10.1073/pnas.1319590110 (2013)).

### Genome and Aging

The nuclear genome contains the vast majority of hereditary information in the cell and its integrity has been found to impact aging process, such as genomic instability, telomere attrition, and the more recent epigenetic alterations (Lopez-Otin et al., Cell 153, 1194-1217, doi:10.1016/j.cell.2013.05.039 (2013)). The first somatic mutation theory of aging dates to 1959 (Szilard, Proc Natl Acad Sci USA 45, 30-45 (1959)). It inferred that the accumulation of somatic mutations inactivates the normal function of vital genes in corresponding tissues, which lead to the organismal decline in aging.

To date, genomic instability has been regarded as one of the hallmarks of aging. Numerous genomic mutations including age-1, daf-2 and daf-16 have been proved to change the lifespan in C. elegant (Tissenbaum, Invertebr Reprod Dev 59, 59-63, doi:10.1080/07924259.2014.940470 (2015)). A mouse model with reporter transgenes also reflected that the frequency of somatic mutation increases with age (Vijg & Dolly, Mech Ageing Dev 123, 907-915 (2002)). Besides, the studies of several human premature aging diseases, such as Bloom syndrome and Werner syndrome, have provided strong evidence that the accelerated accumulation of nuclear mutations leads to progeria (Burtner & Kennedy, Nat Rev Mol Cell Biol 11, 567-578, doi:10.1038/nrm2944 (2010)).

In Werner syndrome, a homozygous mutation of the WRN gene causes a null function of one helicase in the RecQ family. This mutation has a significant impact on DNA transactions, and leads to a large group of somatic mutations. Patients with Werner syndrome usually show a normal phenotype at a young age, but during the time of adolescence, accumulated mutations in the genome lead to a set of symptoms which commonly happen in the aging process, including osteoporosis, diabetes, reduced fertility and an increased predisposition to cancers (Martin & Oshima, Nature 408, 263-266, doi:10.1038/35041705 (2000)). In fact, this increased frequency of DNA mutation has also been reported in normal senescent tissues in human, for instance, lymphocytes and renal tubular epithelial cells (Grist et al., Mutat Res 266, 189-196 (1992), Martin et al., Hum Mol Genet 5, 215-221 (1996)). Moreover, gene edited mouse with improved genetic stability has shown increased healthy longevity and extra resistance to cancer (Vijg & Dollé, Mech Ageing Dev 123, 907-915 (2002)). Hence, it is of interest to study the connection between genomic alteration and aging, with the hope of extending the healthy human lifespan.

### Genomic Instability and Stem Cell Aging

The genomic instability contributes to aging not only by the accumulation of somatic mutations but also by inducing stem cell dysfunction. Stem cell exhaustion has been found in various body compartments with aging in humans, such as the bone (Gruber et al., Exp Gerontol 41, 1080-1093, doi:10.1016/j.exger.2006.09.008 (2006)) and muscle fibers (Conboy & Rando, Cell Cycle 11, 2260-2267, doi:10.4161/cc.20437 (2012)). And studies on mouse have demonstrated that transplantation of young stem cells to the aged mouse will improve the regenerative decline and extend longevity (Lavasani et al., Nat Commun 3, 608, doi:10.1038/ncomms1611 (2012)).

The age-associated DNA damage is believed to be the driver of stem cell aging and eventually results in degenerative changes (Espada & Ermolaeva, Current Stem Cell Reports 2, 290-298, doi:10.1007/s40778-016-0052-6 (2016)). Relative studies have reported that HSCs in normal aged human accumulate hundreds of somatic mutations per genome, and those mutations in turn contribute to HSC aging (Welch et al., Cell 150, 264-278, doi:10.1016/j.cell.2012.06.023 (2012)). The accumulation of mutations in HSCs is also found in normal aged mouse and mouse model harbor deficiencies in DNA damage repair pathway (Rossi et al., Nature 447, 725-729, doi:10.1038/nature05862 (2007)). In fact, stem cells are expected to accumulate more mutations a lifetime since they experience more DNA replication along with cell divisions than most of the other body cells. In Fanconi Anemia, mutations in 19 identified genes lead to a deficiency of DNA repair pathway, resulting in a suppression of hematopoietic stem cell number and function (Ceccaldi et al., Cell Stem Cell 11, 36-49, doi:10.1016/j.stem.2012.05.013 (2012), Duxin & Walter, Curr Opin Cell Biol 37, 49-60, doi:10.1016/j.ceb.2015.09.002 (2015)).

The dysfunction of DNA repair also gives rise to a higher mutation load in somatic cells, which causes a series of symptom as seen during normal aging, such as osteopenia, sarcopenia, and endocrine degeneration (Brosh et al., Ageing Res Rev 33, 67-75, doi:10.1016/j.arr.2016.05.005 (2017)). This provides strong evidence that the high mutation burden and the consequent stem cell dysfunction are correlated to the aging process. It is important to acquire a fundamental understanding of how these mutations get accumulated, which is the earliest stages of aging.

### The Feature of Somatic Mutation—Rare and Uneven Distribution

The aging-related accumulation of genomic mutations is not evenly distributed within the body. Conversely, they result in a heterogeneous cell population with a relatively small proportion of cells with DNA damage. Whole-exome sequencing of DNA in human peripheral-blood cells indicate that clonal expansion of HSCs with somatic mutations exist in 10% of aged people (Genovese et al., N Engl J Med 371, 2477-2487, doi:10.1056/NEJMoa1409405 (2014)).

However, there is still a fundamental gap in understanding the very early stage of HSC aging, which is how these cellular mutations start to accumulate in HSCs, and how these mutations develop with HSC aging. For example, an investigation into cancer development by ultra-deep sequencing (average 500X coverage) of 74 cancer genes from aged tissue was carried out using the Illumina platform (Martincorena et al., Science 348, 880-886, doi:10.1126/science.aaa6806 (2015)). An average of two to six somatic mutations per megabase per cell was observed.

But as introduced above, short-reads based Illumina sequencing is not suitable for detecting rare mutations, especially for surveying rare mutations in a small population of cells. Other studies used clonal cultures of single primary cells followed by whole genome sequencing by Illumina to characterize the dynamic accumulation of mutations in Human Hematopoiesis (Osorio et al., Cell Rep 25, 2308-2316 e2304, doi:10.1016/j.celrep.2018.11.014 (2018), Blokzijl et al., Nature 538, 260-264, doi:10.1038/nature19768 (2016)), but this method can only survey the mutational load in a few cells and whole genome sequence by short-read Illumina with low coverage is short for calling rare mutations. A new strategy with allele-level accuracy is needed for this kind of study.

The development of next-generation sequencing technology has advanced of genomic research in recent years. Precision medicine is one of the most promising frontiers of modern medicine, in which genetic diagnosis by next-generation sequencing (NGS) has been widely used in the clinic. However, shortcomings, such as those mentioned above, make it challenging to use NGS to detect variants and rare mutations (e.g., in a population of cells), which hinders its application, for example, in clinical diagnosis, mitochondrial analysis, stem cell analysis and aging studies, particularly when the mutations are rare or unevenly distributed.

Therefore, there remains a need for new technology to overcome the drawbacks of traditional NGS.

It is an object to the invention to provide compositions and methods for improved nucleic acid sequencing.

### SUMMARY OF THE INVENTION

Compositions and methods for labeling individual nucleic acid (e.g., DNA) molecules with a unique molecular identifier (UMI), followed by amplification by PCR are provided. The PCR amplicons can be grouped by the UMI they contain and traced back to the original molecule. More specifically, the grouped reads with the same UMI represent one original nucleic acid (e.g., DNA) molecule, meaning they share the same nucleic acid sequence.

Methods of sequencing the labeled nucleic acid are also provided. The methods can include determination of a consensus sequence, which thus eliminates errors that may be introduced in the amplification and sequencing process.

Such methods can be used in, for example, the detection of rare genetic variants. By comparing each consensus sequence with the reference sequence, the genetic variations in each original nucleic acid (e.g., DNA) molecule can be detected. Thus, the disclosed method can be used to achieve highly accurate and sensitive nucleic acid sequencing at a single-allele level. These methods are advantageous because UMIs can eliminate errors introduced by PCR amplification so that the accurate sequence of the original DNA molecule can be accurately deduced. Unique molecular identifier (UMI) primers are provided. The UMI primers typically include a universal primer sequence, a unique molecular identifier (UMI) sequence, and a first target nucleic acid binding sequence.

In some embodiments, the orientation of the universal primer sequence, unique molecular identifier (UMI) sequence, and first target nucleic acid binding sequence is 5′ universal primer sequence, unique molecular identifier (UMI) sequence, first target nucleic acid binding sequence 3′.

The universal primer sequence can be any suitable sequence. An exemplary universal primer sequence includes the sequence CATCTTACGATTACGCCAACCAC (SEQ ID NO:1), the reverse sequence thereof, the complementary sequence thereto, the reverse complementary sequence thereof. The UMI sequence can be any suitable sequence (e.g. amenable to bar coding). UMI sequences are usually designed as a string of totally random nucleotides, partially degenerate nucleotides, or defined nucleotides (e.g., when template molecules are limited). The UMI will be sequenced together with the target nucleic acid sequence. UMI sequence can be any NNNN, with variable length, or with any other base (A, T, G, C) inside. For example, a UMI sequence can include NNNNTGNNNN (SEQ ID NO:2), wherein “N” can be A, T, G, or C, the reverse sequence thereof, the complementary sequence thereto, the reverse complementary sequence thereof. The first target nucleic acid binding sequence is designed to bind at or near a gene or other nucleic acid sequence of interest. The first target nucleic acid binding sequence can be designed to bind to genomic or mitochondrial DNA. An exemplary UMI primers is

(SEQ ID NO: 3)
CATCTTACGATTACGCCAACCACTGNNNTGNNNCTCCCGAATCAA
CCCTGACCC

Methods of labeling one or more target nucleic acids are also provided. The methods typically include carrying out at least one cycle of polymerase chain reaction using a first UMI primer on a nucleic acid sample including a nucleic acid sequence to which the first target nucleic acid binding sequence of the primer can bind.

The methods can include a second cycle of PCR further including a second primer alone or in combination with the first primer, the second primer including a second target nucleic acid binding sequence, wherein the target nucleic acid includes a nucleic acid sequence to which the second target nucleic acid binding sequence of the second primer can bind.

Optionally, but preferably, the second primer further includes the same or a different universal primer sequence as the first primer, or the reverse sequence thereof, the complementary sequence thereto, or the reverse complementary sequence thereof. Optionally, but preferably, the second primer further includes the same or different UMI as the first primer, or the reverse sequence thereof, the complementary sequence thereto, or the reverse complementary sequence thereof. For example, the orientation of the universal primer sequence, unique molecular identifier (UMI) sequence, and second target nucleic acid binding sequence of the second primer is 5′ universal primer sequence, unique molecular identifier (UMI) sequence, second target nucleic acid binding sequence 3′. When two UMI primers are used, both ends of the target nucleic acid can be labeled.

In some embodiments, a plurality of sets of first and optionally second UMI primers are used for multiplexing. The nucleic acid binding sequences of each UMI primer set are designed to label the first and optionally second end of a target nucleic acid. The UMI sequence of each primer set can have the same UMI sequence so that different target nucleic acids can be distinguished, but individual molecules of each target nucleic acid cannot necessarily be distinguished by UMI sequence alone. In this way, sequences having the same UMI sequence can be clustered and consensus sequence for each target nucleic acid determined.

Alternatively, the UMI sequence within primers of the primer set can be different UMI sequences so that different target nucleic acids can be distinguished, and individual molecules of each target nucleic acid can also be distinguished by UMI sequence.

The disclosed methods can be used to distinguish small differences (e.g., single nucleotide polymorphisms) among two or more samples (e.g., among two or more genomes, or even alleles).

Optionally, but preferably, third and subsequent rounds (e.g., up to 100) of PCR are carried out to amplify the labeled target nucleic acid(s), optionally using universal primers.

The disclosed methods can be used to one-end or two-end label target nucleic acid(s) with UMI(s). For example, a method of one-end UMI labeling can include a single round of extension of a UMI primer including a universal primer sequence, unique molecular identifier sequence, and target nucleic acid binding sequence that hybridizes to a target nucleic acid sequence and optionally removing the UMI primer from the reaction mixture. A method of two-end UMI labeling can include a single round of extension of a forward UMI primer including a universal primer sequence, unique molecular identifier sequence, and target nucleic acid binding sequence that hybridizes to a target nucleic acid sequence and optionally removing the forward UMI primer from the reaction mixture, and a single round of extension of a reverse UMI primer including a universal primer sequence, unique molecular identifier sequence, and target nucleic acid binding sequence that hybridizes to a target nucleic acid sequence and optionally removing the reverse UMI primer from the reaction mixture. Next, the one- or two-end labeled target nucleic acids can be amplified by PCR with a universal primer alone or in combination with a target nucleic acid specific primer, wherein the cycles of PCR amplify the one- or two-end UMI labeled target nucleic acid. Exemplary embodiments are illustrated in FIGS. 8, 9A, and 12A.

In some embodiments, the nucleic acid sample is nuclear genomic DNA, mitochondrial genomic DNA, or a combination thereof. The source of the nucleic acid sample can be, for example, any integer between 1 and 1,000,000 cells inclusive, or any range formed of two integers there between, for example, between 1 and 10,000, 1 and 1,000, 1 and 100, 1 and 10, or 1 single cell. In some embodiments, the source of the nucleic acid sample is one single nuclei or one single mitochondrion. In some embodiments, the nucleic acid sample is isolated from a cell or cells. Isolation can include releasing the target nucleic acid sample by lysing the cell(s). Some embodiments include removing contaminants (e.g., one or more of primers, dNTPs, RNA, etc.), before the first cycle of PCR, after the first cycle of PCR, after the last cycle of PCR, or any combination thereof.

Methods of determining the sequence of a target nucleic acid are also provided and can include, for example,
(i) labeling one or more target nucleic acids;
(ii) sequencing the labeled amplicons;
(iii) optionally grouping sequences having the same UMI into one of more groups;
(iv) determining the sequence of each target nucleic acid sequence by determining the consensus sequence of each group.

Some embodiments include (v) identifying polymorphisms in one or more of the target nucleic acids. The polymorphism can be a single nucleotide polymorphism (SNP).

In some embodiments, the sequencing includes long-read sequencing technology. In some embodiments, the long-read sequencing technology includes a Nanopore MinION sequencer. In some embodiments, the long-read sequencing technology includes preparing a 1D ligation library from the labeled amplicons.

Any of steps (iii)-(v) can be carried out using bioinformatics analysis. In some embodiments, the bioinformatics analysis includes basecalling, sequence alignment(s), polymorphism identification or a combination thereof.

Another method of labeling a target nucleic acid and optionally sequencing the labeled target nucleic includes

(i) restriction enzyme (e.g., BsrG1) digest of only the nuclear DNA in a nucleic acid sample including nuclear and mitochondrial DNA;

(ii) treatment of the nucleic acid sample with lambda exonuclease;

(iii) labeling of the remaining mtDNA with UMI labels, priming sites, and bar codes using EZ-Tn5 transposon;

(iv) sequencing the labeled mtDNA.

Any of the methods can further include amplifying the nucleic acid sample, or a fraction thereof, prior to labeling.

Any of the methods can further include one or more rounds of enrichment and/or purification of the nucleic acid sample, target nucleic acid, amplicons, or otherwise labeled nucleic acid, including, for example, size selection.

### BRIEF DESCRIPTION OF THE DRAWINGS

FIG. 1A is a schematic of using UMIs to label individual DNA molecule in a cell and illustrates how PCR errors are eliminated by grouping reads based on UMIs. FIG. 1B is a schematic of PCR-directed single DNA labeling with two-end UMIs. FIG. 1C is a schematic of individual DNA molecule labeling illustrated on a circular nucleic acid such as mitochondrial DNA (mtDNA). FIG. 1D is a photograph of an electrophoretic gel showing the 16.5 kb of full-length mtDNA are amplified using optimized PCR. FIG. 1E is a photograph of an electrophoretic gel showing mtDNA from purified 293T genome labeled using the aforementioned method (label lane) without non-specific amplification (using only universal primers to amplify genome, control lane).

FIGS. 2A-2D illustrate alignment and length distribution of reads generated by Nanopore MinION. FIG. 2A is a plot showing reads from a 16.5 kb amplicon sequencing mapped to the human mitochondrial genome.

FIGS. 2B-2D are plots showing the length distribution of reads from amplicon sequencing. Long-length peaks from left to right are 7.7 kb, 8.6 kb, 11 kb, 11.9 kb, 12.7 kb, and 16.5 kb.

FIGS. 3A-3C illustrate the establishment of a data-analysis pipeline. FIG. 3A is a bar graph showing a comparison of three alignment algorithms, graphmap, minimap2 and bwa-mem. FIG. 3B is a plot of the data set used for evaluating SNPs-calling algorithms Three homozygous SNPs identified by Sanger sequencing are shown with respective coverage.

FIG. 3C is a flow chart showing a pipeline for data analysis. Raw fast5 reads are basecalled by albacore, followed by trimming adapter using porechop. Refined fastq reads are mapped to reference using graphmap, subsequently analyzed by samtools to call SNPs.

FIGS. 4A-4E illustrates mtDNA labeling from one hundred of 293T cells. FIG. 4A is a schematic of a work flow for labeling mtDNA with UMIs from cells. FIG. 4B is a schematic of PCR-directed single DNA labeling with single-end UMIs. FIG. 4C is an electrophoretic gel showing 16.5 kb of UMIs labeled mtDNA are generated using the strategy shown in FIG. 4A. FIG. 4D is an electrophoretic gel showing small fragments are eliminated by BluePippin, while they remain after AMPure purification. FIG. 4E is an electrophoretic gel showing label mitochondrial DNA with UMIs in 50, 25, and 10 cells. FIG. 4F is a schematic illustrating a strategy used to extract UMIs. 3478 of unique UMIs are found. (SEQ ID NOS:18-21).

FIG. 5 is a schematic using EZ-Tn5 transposase to label individual mtDNA.

FIG. 6 is a flow chart showing an experimental design for analyzing mtDNA mutations during development and aging.

FIG. 7 is a schematic of an experimental design for analyzing the mutational processes in HSC aging in mouse.

FIG. 8 is a schematic representation showing steps utilized in some embodiments of the disclosed methods, and a particular embodiment also referred to in Example 5 as IDMseq (center workflow) contrasted with ligation of UMI adaptors (left side workflow) and PCR-directed UMI labeling (right side workflow), and analyzed by VAULT (center workflow) contrasted with UMI analysis by clustering algorithms (right side workflow). A given population of cells (symbolized by dotted oval) may contain different alleles of a target locus, which accounts for a small proportion of the pool of genomic DNA. The first step of targeted molecular consensus sequencing is labeling of the variant alleles with UMI. Ligation-based and PCR-directed UMI labeling are two alternative methods. Ligation-based UMI labeling will label irrelevant regions and the low efficiency of ligation will also omit a proportion of target alleles (greyed out in the middle left panel). PCR-directed UMI labeling is highly efficient but will result in UMI clashes (one original molecule labeled with multiple UMIs, leading to false UMI groups, middle right panel). IDMseq is the only method with high labeling efficiency and can faithfully retain the allele information (variants and frequency). After UMI labeling, the DNA with UMIs are amplified for sequencing in appropriated platforms (e.g., Illumina, Nanopore or PacBio). In the data analysis step, the algorithm needs to identify reads with the same UMI and use these to get the consensus sequence of the allele. This step can be done with read-clustering algorithms that work well for fixed-length reads of short-read sequencing (e.g. Illumina). However, this strategy could miss reads with complex changes such as those uncovered by long-read sequencing, which prevents detection of deletions, insertions and complex structural variants (lower left panel). VAULT performs a BLAST-like strategy to locate UMI sequence in reads regardless of length and structure. VAULT analysis thus preserves the sequence information of all types of alleles and their frequency (lower middle and right).

FIG. 9A is a schematic representation showing steps utilized for UMI labeling in some embodiments of the disclosed methods, and differences therein for one-end verse two-end UMI labeling. UMI primers are used to label individual DNA molecules with unique UMIs (one molecule is labeled with one UMI). It contains a 3′ gene-specific sequence, a UMI sequence, and a 5′ universal primer sequence. The 3′ gene-specific sequence is selected for its high specificity to the target gene. The middle UMI sequence contains multiple random bases (denoted by Ns). The 5′ universal primer sequence is used to uniformly amplify all UMI-tagged DNA molecules. Some of the disclosed embodiments including IDMseq are different from other UMI-based methods in that barcoding is achieved by a single round of primer extension rather than multiple cycles of PCR as commonly practiced (Kinde et al., Proc Natl Acad Sci U S A 108, 9530-9535 (2011), Hiatt et al., Nat Methods 7, 119-122 (2010)). For two-ended labeling, an additional round of primer extension with reverse UMI primers will be done after removing forward UMI primers. The UMI-labeled DNA will be further amplified by universal primers before sequencing. FIG. 9B is a flowchart showing an exemplary pipeline for data analysis. This embodiment is also referred to herein and in Example 5 as VAULT analysis. During data per-processing, raw reads were filtered and mappable reads were extracted. After that, VAULT applies a BLAST-like strategy to locate UMI sequence in reads by searching for the known sequences of the universal primer and gene-specific forward primer. After that, VAULT bins reads according to UMI. The last steps of VAULT are variant calling for both SNVs and large SVs and report generation.

FIG. 10A is a schematic representation of an experimental design utilized in the experiments of Example 5. Cas9 RNP and ssODN were electroporated to H1 ESCs to generate homozygous G>A single-base substitution in the EPOR gene. FIG. 10B is a schematic of the Cas9 target site and the Ncol restriction site. A restriction enzyme digestion assay was used to identify the knock-in hESC clones. Wild-type EPOR gene contains a Ncol site and thereby can be digested. The Knock-in allele will lose the Ncol site and cannot be digested. (SEQ ID NO:22-23).

FIGS. 11A-11C are aligned read length vs. percent identity plot using kernel density estimation for Nanopore sequencing of the 1:10,000 population, Illumina sequencing of the 1:10,000 population, PacBio sequencing of the 1:1,000 population.

FIG. 12A is a schematic representation showing steps utilized in some embodiments of the disclosed methods, and a particular embodiment also referred to in Example 5 as IDMseq. Individual DNA molecules are labeled with unique UMIs and amplified for sequencing on appropriate platforms (e.g. Illumina, PacBio, and Nanopore). During data analysis, reads are binned by UMIs to correct errors introduced during amplification and sequencing. Both SNV and SV calling are included in the analysis pipeline. FIG. 12B is an illustration showing examples of Integrative Genomics Viewer (IGV) tracks of UMI groups in which the spike-in SNV in the 1:10000 population was identified by IDMseq and VAULT. The knock-in SNV is indicated by the triangle in the diagram of the EPOR gene on top, and also shown as “T” base in the alignment map. The gray bars show read coverage. The ten colored bars on the left side of the coverage plot represent the UMI sequence for the UMI group. Individual Nanopore (top) and Illumina (bottom) reads within the group are shown under the coverage plot. FIG. 12C is an illustration of showing large SVs detected by IDMseq in the 1:1000 population on the PacBio platform. Three UMI groups are shown with the same 2375 bp deletion. Group 1 represents one haplotype, and Group 2&3 represent a different haplotype. Colored lines represent the SNPs detected in each group. Thick boxes: exons; thin boxes UTRs. Thin vertical lines in the gene diagram represent PCR primer location. FIG. 12D is a plot showing distribution of SNVs detected by PacBio sequencing in conjunction with IDMseq and VAULT. One of the SNVs was also found in the Nanopore dataset. The spike-in SNV (1:1000) is indicated by the triangle. FIG. 12E is a plot showing the frequency distribution of the variant allele fraction of SNVs detected by IDMseq in PacBio sequencing of the EPOR locus. FIG. 12F is a chart showing the spectrum of base changes among somatic SNVs. The majority of base changes are G to A and C to T. FIG. 12G is a plot showing comparison between observed VAF and expected VAF in different experiments and sequencing platforms.

FIG. 13A is a schematic representation of an experimental design utilized in Example 5. Cas9 RNPs designed to cleave the first exon of PANXJ were electroporated to H1 hESCs. IDMseq was used to analyze the locus in edited hESCs 48 hours later. FIG. 13B is an aligned read length vs. percent identity plot using kernel density estimation of Nanopore sequencing data of a 7077 bp region encompassing the Cas9 cleavage. FIG. 13C is an illustration of large SVs detected by IDMseq and VAULT in edited hESCs. Five SV groups were shown with deletion length ranging from 270 bp to 5494 bp. The dotted line represents the Cas9 cutting site. The coverage of Nanopore reads is shown on top of each track in gray. The colored lines on the left side of the coverage plot represent the UMI for the group. Individual Nanopore reads within the group are shown under the coverage plot. FIG. 13D is a plot showing distribution of SNVs detected by IDMseq and VAULT in edited hESCs. Somatic SNVs and cell-line specific SNVs are shown. Somatic SNVs cannot be detected if variant calling is done en masse without UMI analysis (see the coverage track). Cell-line specific SNVs are detected in ensemble analysis (see colored lines in the coverage track) and most of them have been reported as common SNPs in dbSNP-141 database (Common SNPs track). The Cas9 cut site is indicated by a triangle. FIG. 13E is a chart showing analysis of somatic mutations detected in CRISPR-edited hESCs based on base change. The majority of base changes are G to A and C to T.

FIG. 14A is an aligned read length vs. percent identity plot using kernel density estimation of Nanopore sequencing data of a 6595 bp region encompassing the Cas9 cleavage. FIG. 14B-14C are alignments of individual alleles from Sanger sequencing of single-cell derived hESC clones after Cas9-directed mutagenesis in exon 1 of PANX1 using Pan1 sgRNA (14B (SEQ ID NOS:24-40)) or Pan3 sgRNA (14C (SEQ ID NOS:41-49)). The gRNA sequence (Pani, CGGAGTACGTGTTCTCGGAT (SEQ ID NO:6); Pan 3, CTGTTCTGGCGTTTCGCAGC (SEQ ID NO:7)) are shown and the cleavage site is indicated by a dotted line. Texts indicate insertion or deletion events.

FIG. 15A is a plot showing that the frequency of deletions or insertions of different size detected in Pan1-edited hESCs. Certain deletions and insertions occur at disproportionally high frequencies. For example, a 5494 bp deletion was found in 56 UMI groups, which indicates a possible hotspot of Cas9-induced large deletion. FIG. 15B is a plot showing the frequency of different size deletions or insertions detected in Pan3-edited hESCs. Certain deletions and insertions occur at disproportionally high frequencies. For example, a 4238 bp deletion was found in 27 UMI groups, which indicates a possible hotspot of Cas9-induced large deletion. FIGS. 15C-15D are plots showing the frequency distribution of the variant allele fraction of SNVs detected by IDMseq in Nanopore sequencing of the PANX1 locus in Pan1-edited hESCs (15C), and Nanopore sequencing of the PANX1 locus in Pan3-edited hESCs (15D). FIG. 15E is a chart showing analysis of somatic mutations detected in Pan3-edited hESCs based on base change. The majority of base changes are G to A and C to T.

### DETAILED DESCRIPTION OF THE INVENTION

### I. Definitions

“Isolated,” “isolating,” “purified,” “purifying,” “enriched,” and “enriching,” when used with respect to nucleic acids of interest (e.g., DNA such as intact or fragmented genomic DNA, amplicons, etc.,), indicate that the nucleic acids of interest at some point in time were separated, enriched, sorted, etc., from or with respect to other cellular material to yield a higher proportion of the nucleic acids of interest compared to the other cellular material, contaminates, or active agents such as enzymes, proteins, detergent, cations or anions. “Highly purified,” “highly enriched,” and “highly isolated,” when used with respect to nucleic acids of interest, indicates that the nucleic acids of interest are at least about 70%, about 75%, about 80%, about 85%, about 90% or more, about 95%, about 99% or 99.9% or more purified or isolated from other cellular materials, contaminates, or active agents such as enzymes, proteins, detergent, cations or anions. “Substantially isolated,” “substantially purified,” and “substantially enriched,” when used with respect to nucleic acids of interest, indicate that the nucleic acids of interest are at least about 70%, about 75%, or about 80%, more usually at least 85% or 90%, and sometimes at least 95% or more, for example, 95%, 96%, and up to 100% purified or isolated from other cellular materials, contaminates, or active agents such as enzymes, proteins, detergent, cations or anions.

As used herein, the term “amplicon” refers to product of amplification, for example, polymerase chain reaction (PCR). “Amplicons” can refer to a homogenous plurality of amplicons, for example a specific amplification product, or a heterogenous plurality of amplicons, for example a non-specific or semi-specific amplification product.

As used herein, the term “restriction endonuclease” or “restriction enzyme” or “RE enzyme” is any enzyme that recognizes one or more specific nucleotide target sequences within a DNA strand, to cut both strands of the DNA molecule at or near the target site.

As used herein, the term “nucleotide” and “nucleic acid” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, the terms “oligonucleotide” or a “polynucleotide” are synthetic or isolated nucleic acid polymers including a plurality of nucleotide subunits.

With respect to the disclosed polynucleotide sequences, “N” can be any nucleotide (e.g., A or G or C or T), “R” can be any purine (e.g., G or A), and Y can be any pyrimidine (e.g., C or T).

As used herein, the terms “complement”, “complementary”, and “complementarity” with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′”. The second sequence can be referred to as the reverse complement of the first sequence, and the first sequence can be referred to as the reverse complement of the second sequence.

Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, the term “substantially complementary” means that two sequences hybridize. In some embodiments, the hybridization occurs only under stringent hybridization conditions. The skilled artisan will understand that substantially complementary sequences can, but need not allow, hybridize along their entire length. In particular, substantially complementary sequences may comprise a contiguous sequence of bases that do not hybridize to a target sequence, positioned 3′ or 5′ to a contiguous sequence of bases that hybridize e.g., under stringent hybridization conditions to a target sequence.

As used herein, the term “hybridize” refers to a process where two substantially complementary or complementary nucleic acid strands anneal to each other under appropriately stringent conditions to form a duplex or heteroduplex through formation of hydrogen bonds between complementary base pairs.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary or complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are between about 15 to about 60 nucleotides in length, and or between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “primer pair” or “primer set” refers to a forward and reverse primer pair (i.e., a left and right primer pair) that can be used together to amplify a given region of a nucleic acid of interest. As used herein, the term “polymorphism” means variations of a nucleotide sequence in a population. For example, polymorphism can be one or more base changes, an insertion, a repeat, or a deletion. Polymorphisms can be single nucleotide polymorphisms (SNP), or simple sequence repeat (SSR). SNPs are variations at a single nucleotide, e.g., when an adenine (A), thymine (T), cytosine (C) or guanine (G) is altered. Generally a variation must generally occur in at least 1% of the population to be considered a SNP.

As used herein, the terms “aligning” and “alignment” refer to the comparison of two or more nucleotide sequence based on the presence of short or long stretches of identical or similar nucleotides. Several methods for alignment of nucleotide sequences are known in the art, as will be further explained below.

As used herein, the term “subject” includes, but is not limited to, animals, plants, bacteria, viruses, parasites and any other organism or entity. The subject can be a plant. The subject can be an animal, such as a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. A cell can be in vitro. Alternatively, a cell can be in vivo and can be found in a subject. A “cell” can be a cell from any organism including, but not limited to, a bacterium.

### II. Compositions and Methods for Nucleic Acid Sequence Analysis

A. Primers for Labeling Target Nucleic Acid Sequences

Compositions and methods for labeling targeting nucleic acid sequences are provided. The methods typically rely on one or more cycles of PCR with one or more primers at least one of which is a unique molecular identifier (UMI) primer. As used herein, bind and hybridize are used interchangeably to refer to the desired interaction between a PCR primer and the nucleic acid it targets for amplification.

A unique molecular identifier (UMI) primer typically includes one or more of a universal primer sequence, a unique molecular identifier (UMI) sequence, and a first target nucleic acid binding sequence. The orientation of the primer elements can be, for example, 5′ universal primer sequence, unique molecular identifier (UMI) sequence, first target nucleic acid binding sequence 3′. The universal primer sequence is one that serves as a binding site for a universal primer once the universal primer sequence(s) is incorporated onto the end or ends of a target nucleic acid (e.g., universal primer sequence labeled). The universal primer sequence(s), when flanking the target nucleic acid, allow for multiplexed amplification of numerous, and uniquely labeled (e.g., UMI sequence labeled) target nucleic acids using a single primer set in a single PCR reaction. The universal primer sequence can be any suitable length and sequence. In some embodiments, the universal primer sequence is designed so that the same, single universal primer can amplify target nucleic acid(s) flanked by universal primer sequences. Thus, the universal primer set may be only a single primer that works as both a forward and reverse primer.

In a preferred embodiment, a universal primer sequence includes the sequence CATCTTACGATTACGCCAACCAC (SEQ ID NO:1), or the reverse sequence thereof, the complementary sequence thereto, or the reverse complementary sequence thereof.

The UMI sequence provides a unique molecular identity to the target the nucleic acid once the UMI sequence is incorporated onto the target nucleic acid (e.g., UMI sequence labeled). UMI sequences are usually designed as a string of totally random nucleotides (such as NNNN or NNNNNNN), partially degenerate nucleotides (such as NNNRNYN or NNNNTGNNNN (SEQ ID NO:2)), or defined nucleotides (e.g., when template molecules are limited). The UMI will be sequenced together with the target nucleic acid sequence. After sequencing, the reads can optionally be sorted by UMI and grouped together (i.e., demultiplexing).

UMI sequences can be or include any NNNN, with variable length, or with any other base (A, T, G, C) inside. UMI sequences are not limited to the sequences utilized in the Examples below, i.e. NNNNTGNNNN (SEQ ID NO:2). UMI sequences can be of any length of nucleotides with any sequence, for example between about 5 nucleotides to about 100 nucleotides (e.g., “N's”).

In an exemplary embodiment, the UMI sequence includes NNNNTGNNNN (SEQ ID NO:2), wherein “N” can be A, T, G, or C, or the reverse sequence thereof, the complementary sequence thereto, or the reverse complementary sequence thereof.

Typically, the first target nucleic acid binding sequence binds (hybridizes) at or near a first site in the target nucleic acid sequence of interest, for example a gene of interest. The target nucleic acid binding allows for specific labeling (e.g., universal primer labeling, UMI labeling, or the combination thereof) and/or amplification of the target nucleic acid.

In embodiments, the first target nucleic acid binding sequence binds to nuclear DNA or mitochondrial DNA (mtDNA).

In a particular embodiment, a UMI primer for binding mtDNA includes

(SEQ ID NO: 3)
CATCTTACGATTACGCCAACCACTGNNNTGNNNCTCCCGAATCAA
CCCTGACCC.

A second primer typically includes a second target nucleic acid binding sequence that can bind to a second site in the target nucleic acid sequence of interest, for example a gene of interest. The second primer can be a second UMI primer.

The second target nucleic acid primer can optionally include the same or a different UMI sequence as the first primer, and can optionally include the same or a different universal primer sequences as the first primer. The orientation of the primer elements can be, for example, 5′ universal primer sequence, unique molecular identifier (UMI) sequence, first target nucleic acid binding sequence 3′.

The first and second primers are designed to flank the target nucleic acid sequence and label one or both ends with the universal primer sequence(s), UMI sequence(s), or combination thereof. The first and second primers may also be used to amplify the target nucleic acid.

Each of the universal primer sequence(s), the UMI sequence(s), and the target nucleic acid binding sequence(s) can include any number/length of nucleotides having any sequence suitable to achieve its molecular identifier and/or priming function(s). For example, in some embodiments, one or more of the universal primer sequence, the UMI sequence, and the target nucleic acid binding sequence of each primer has between about 5 and about 100 nucleotides, respectively. In some embodiments, one or more of one or more of the universal primer sequence, the UMI sequence, and the target nucleic acid binding sequence of each primer has any specific integer number of nucleotides between 5 and 100 nucleotides, inclusive, or range between two integers there between, respectively.

Any of the disclosed primers, including first and second UMI primers, first and second universal primers, or any combination thereof, can include any number/length of nucleotides having any sequence suitable to achieve its molecular identifier and/or priming function(s). For example, in some embodiments, one or more of UMI and/or universal primers have between about 5 and about 100 or about 500 nucleotides. In some embodiments, one or more of the UMI and/or universal primers have any specific integer number of nucleotides between 5 and 500 nucleotides, inclusive, or range between two integers there between.

In some embodiments, a plurality of sets of first and optionally second UMI primers are used for multiplexing. The nucleic acid binding sequences of each UMI primer set are designed to label the first and optionally second end of a target nucleic acid. The UMI sequence of each primer set can have the same UMI sequence so that different target nucleic acids can be distinguished, but individual molecules of each target nucleic acid cannot necessarily be distinguished by UMI sequence alone. In this way, sequences having the same UMI sequence can be clustered and consensus sequence for each target nucleic acid determined.

Alternatively, the UMI sequence within primers of the primer set can be different UMI sequences so that different target nucleic acids can be distinguished, and individual molecules of each target nucleic acid can also be distinguished by UMI sequence.

The UMI primers may further include a sample bar code. The sample bar code is unique to each sample, but not each target nucleic acid. The sample bar code can follow the same general guidelines provided herein for designing UMI sequences.

Preferably, the universal primer sequence, UMI sequence, target nucleic acid sequence, and sample bar code can be distinguished.

B. Methods of Labeling a Target Nucleic Acid

Methods of labeling a target nucleic acid are provided. Typically, the first primer alone or in combination with the second primer can be used during one or more PCR cycles to amplify a fragment of the nucleic acid sample that includes or consists of the target nucleic acid sequence or a fragment thereof. The nucleic acid sample serves as the initial template for this PCR. The amplified fragment can be referred to as an amplicon.

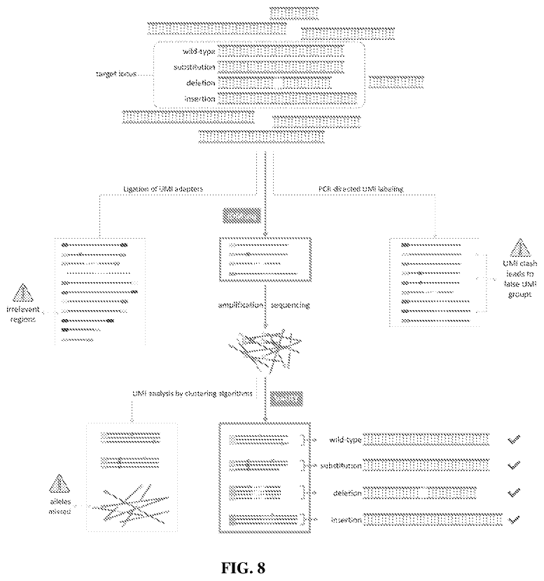

A given population of cells may contain different alleles of a target locus, which accounts for a small proportion of the pool of genomic DNA. A first step of targeted molecular consensus sequencing is labeling of the variant alleles with UMI. Ligation-based and PCR-directed UMI labeling are two widely used methods. However, ligation-based UMI labeling will label irrelevant regions and the low efficiency of ligation will also omit a proportion of target alleles (see, e.g., FIG. 8). PCR-directed UMI labeling is highly efficient but will result in UMI clashes (one original molecule labeled with multiple UMIs, leading to false UMI groups). The disclosed methods can be used to achieve high labeling efficiency and can faithfully retain the allele information (variants and frequency). After UMI labeling, the DNA with UMIs are amplified for sequencing in appropriated platforms (Illumina, Nanopore or PacBio, etc.).

Thus, the methods typically include carrying out at least one cycle of polymerase chain reaction using a first UMI primer, such as those introduced above, on a nucleic acid sample including a nucleic acid sequence to which the first target nucleic acid binding sequence of the first UMI primer can bind.

In some embodiments, the methods include carrying out at least one cycle of polymerase chain reaction using a plurality of different first UMI primers, such as those introduced above, on a nucleic acid sample including nucleic acid sequences to which a plurality of first target nucleic acid binding sequences of the first UMI primers can bind (e.g., a multiplex reaction that labels a first end of two or more target nucleic acids depending on the number of first UMI primers used).

In some embodiments, the UMI sequence for each first UMI primer includes one UMI sequence matched to one target nucleic acid binding sequence, thus each individual molecule of the target nucleic acid is labeled with the same UMI sequence, but each different nucleic acid target is labeled with a different UMI. In this way, different nucleic acid targets can be distinguished, but not necessarily different individual molecules (e.g., the same target in two different genomes) based on UMI alone.

In some embodiments, the UMI sequence for each first UMI primer includes different or unique UMI sequences matched to one target nucleic acid binding sequence, thus each individual molecule of the target nucleic acid is labeled with the a different UMI sequence, and each different nucleic acid target is labeled with a different UMI. In this way, different nucleic acid targets can be distinguished, and different individual molecules can also be distinguished based on UMI alone.

In some embodiments, the at least one cycle of polymerase chain reaction cycle of PCR further includes a second primer, as introduced above, including a second target nucleic acid binding sequence and the target nucleic acid includes a nucleic acid sequence to which the second target nucleic acid binding sequence of the second primer can bind. In some embodiments, the first cycle of PCR does not include a second primer.

In some embodiments, a second and optionally one or more subsequent cycles of PCR includes a second primer and optionally the first primer. Thus, in some embodiments, the first cycle is carried with the first primer alone or both the first and a second primer; and the second and/or subsequent cycles are carried out with a second primer alone, or with both the first and second primers. In some embodiments, all cycles of PCR are carried out with both a first and a second primer. Thus, in some embodiments, the first, second, and subsequent PCR cycles are all the same. In some embodiments, the first and second PCR cycles are different.

As introduced above, the second primer can further include the same or a different universal primer sequence as the first primer, or the reverse sequence thereof, the complementary sequence thereto, or the reverse complementary sequence thereof. The second primer can further include the same or different UMI as the first primer, or the reverse sequence thereof, the complementary sequence thereto, or the reverse complementary sequence thereof. In some embodiments, the second primer does not include a universal primer sequence, and/or does not include a UMI. In some embodiments, the second primer consists only of a second target nucleic acid binding sequence.

In some embodiments, the methods include carrying out at least one cycle of polymerase chain reaction (the second total cycle) using a plurality of second UMI primers, such as those introduced above, on a nucleic acid sample including nucleic acid sequences to which a plurality of second target nucleic acid binding sequences of the second UMI primers can bind (e.g., a multiplex reaction that labels a second end of two or more target nucleic acids depending on the number of second UMI primers used).

In some embodiments, the UMI sequence for each second UMI primer includes one UMI sequence matched to one target nucleic acid binding sequence, thus each individual molecule of the target nucleic acid is labeled with the same UMI sequence, but each different nucleic acid target is labeled with a different UMI. In this way, different nucleic acid target can be distinguished, but not necessarily different individual molecules (e.g., the same target in two different genomes) based on UMI alone. The UMI sequence of the second UMI primer can be the same or different from the UMI sequence of the first UMI primer.

In some embodiments, the UMI sequence for each second UMI primer includes different or unique UMI sequences matched to one target nucleic acid binding sequence, thus each individual molecule of the target nucleic acid is labeled with the a different UMI sequence, and each different nucleic acid target is labeled with a different UMI. In this way, different nucleic acid targets can be distinguished, and different individual molecules can also be distinguished based on UMI alone. The UMI sequence of the second UMI primer can be the same or different from the UMI sequence of the first UMI primer.

The first and second target nucleic acid binding sequences of the primer sets are designed to flank the target nucleic acid region so that it can be amplified using subsequent rounds of amplicon amplification, preferably using universal primers.

The method can include zero, or any integer number of second and subsequent PCR cycles, for example between 1 and 100 inclusive subsequent cycles of PCR.

Thus in some embodiments, the synthetic DNA also referred to as amplicons generated by the first and/or the second or subsequent PCR cycles includes one or both ends labeled with one or more of a universal primer sequence, a UMI, or the combination thereof.

In particular embodiments, the nucleic acid sample is amplified by two rounds of one-cycle PCR with respective (e.g., first and second) UMI-containing primers. After that, two universal primers are used to amplify the labeled amplicons.

As introduced above, one or more first primers alone or in combination with one or more second primers can be used separately or together to amplify two or more different target sequence amplicons. In some embodiments, different amplicons generated during separate PCR reactions are combined prior to amplicon amplification and/or sequencing.

In preferred embodiments, subsequent to one or more cycles of PCR using the UMI primer(s), a new one or more cycles of PCR are carried out using primer(s) that bind to the universal primer sequence and further amplify the amplicons. Typically, the template for this PCR is or includes the amplicons that include one or more UMI sequences and one or more universal primer sequences. Preferably, the amplicon has both ends labeled with the same or different universal primer sequences. In some embodiments, two or more different amplicons containing different nucleic acid target sequences contain the same universal primer sequence and different UMI sequences and can be amplified together using the same universal primers.

In some embodiments, the UMI primers are designed so that the first and second (e.g., forward and reverse) universal primers have the same sequence.

In some embodiments, such as when only one end of the target nucleic acid is label, the amplicon amplification can be carried out with one universal primer, and one random or target nucleic acid specific primer.

Any integer number of amplicon amplification PCR cycles can be carried out, for example, between 1 and 100 inclusive cycles of PCR including primers that bind to the one or more universal primer sequences. The number of cycles can depend on the abundance of the target sequence.

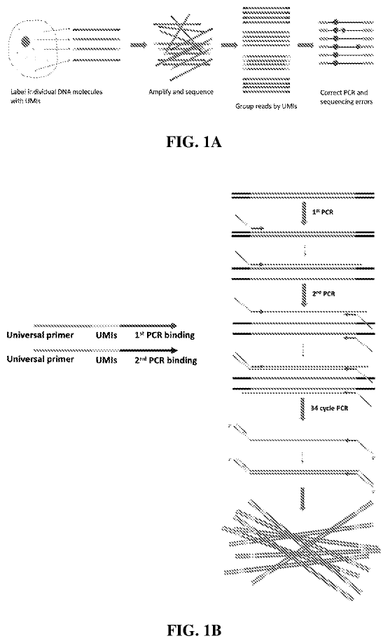

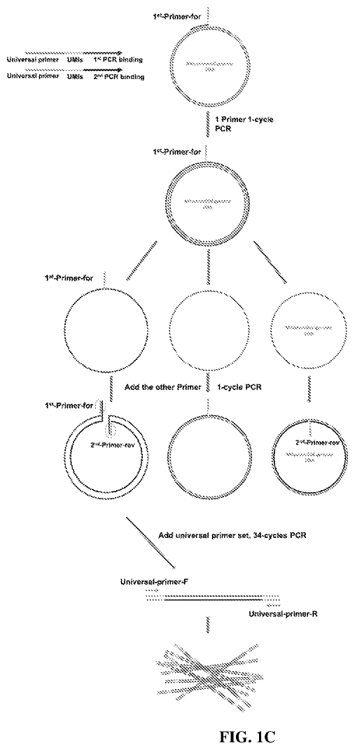

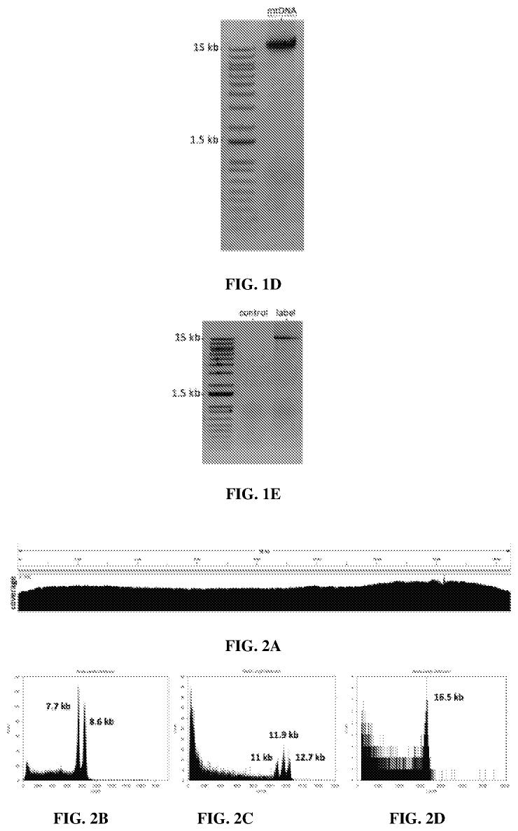

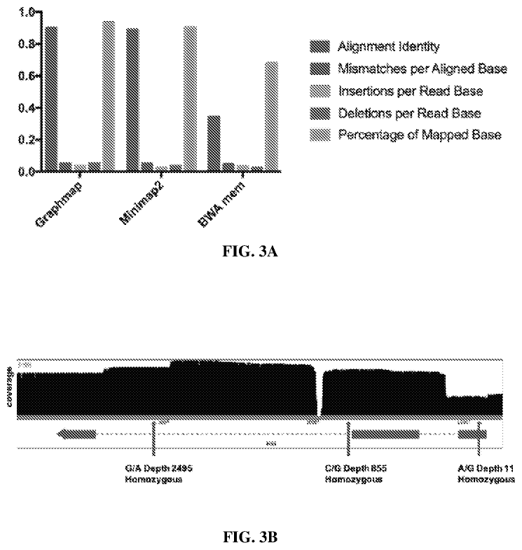

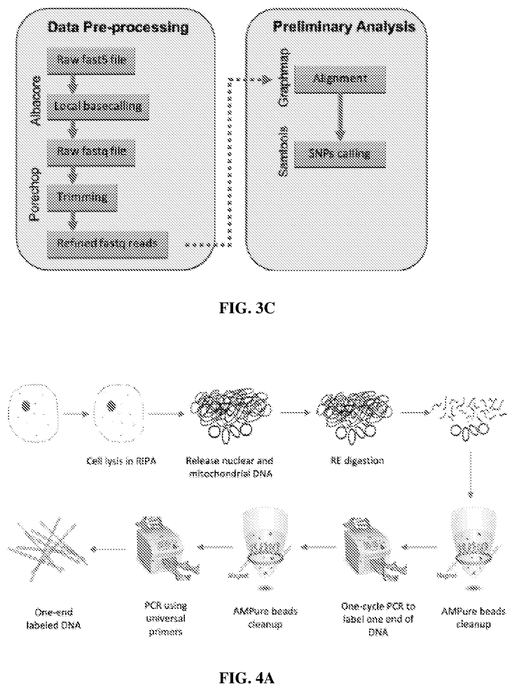

In some embodiments, the disclosed methods include one or more steps of any of FIGS. 1A, 1B, 1C, 3C 4A, 4B, 4F, 8, 9A, 9B, and/or 12A.

The PCR step(s) typically includes an effective amount of the desired primer to accomplish the intended goal of adding a label and/or amplifying an amplicon.

In particularly preferred embodiments, the nucleic acid sample is amplified by two rounds of one-cycle PCR with respective (e.g., first and second) UMI-containing primers, or sets thereof. The first one-cycle PCR (e.g., extension of first primer) adds a universal primer sequence and UMI sequence to one end of a target nucleic acid. The second one-cycle PCR (e.g., extension of at least second primer, and optionally first primer) adds a universal primer sequence and UMI sequence to the other end of the target nucleic acid. As discussed herein, this first and second one-cycle PCRs may include a plurality of different first and second UMI primers (i.e., primer sets), respectively, that allow simultaneous (e.g. multiplex) labeling of a plurality of different target nucleic acids. Next, two universal primers can be used to amplify the labeled amplicons, which may include one target nucleic acid or a plurality of different target nucleic acids.

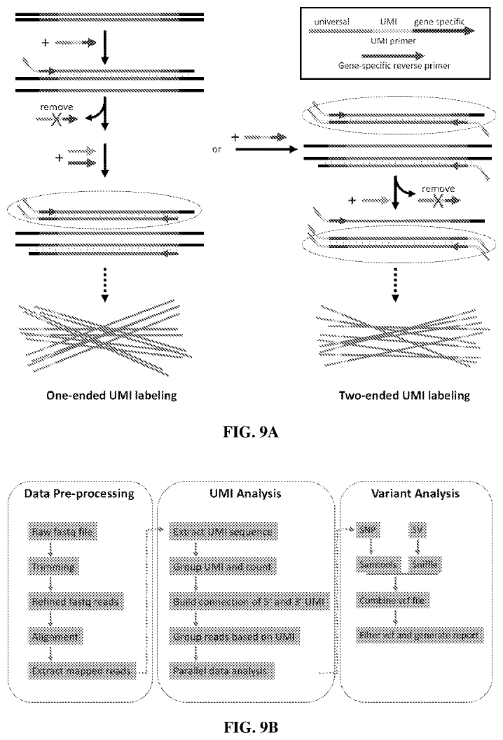

FIG. 9A is schematic representation of two particularly preferred embodiments of UMI labeling and target nucleic acid amplification: one-end UMI labeling (left side) and two-end UMI labeling (right side). As discussed herein, UMI primers are first used to label individual DNA molecules with unique UMIs (one molecule is labeled with one UMI). As depicted in FIG. 9A, in some embodiments, one-end UMI labeling includes or consists of one cycle of PCR with a UMI primer to UMI label one end of the target nucleic acid, followed by one or more cycles of PCR amplification using a universal primer in combination with a target nucleic acid specific primer. In some embodiments, two-end UMI labeling includes or consists of one cycle of PCR with a UMI primer to label one end of the target nucleic acid, followed by one cycle of PCR with e.g., a second UMI primer to label the other end of the target nucleic acid, followed by one or more cycles of PCR amplification using e.g., a universal primer.

Suitable UMI primers are described above and can contain, e.g., a 3′ genes-specific sequence, a UMI sequence, and a 5′ universal primer sequence. The 3′ gene-specific sequence is selected for its high specificity to the target gene. The middle UMI sequence typically includes multiple random bases (denoted by Ns). The 5′ universal primer sequence is used to uniformly amplify all UMI-tagged DNA molecules.

Preferred embodiments of the disclosed methods are different from other UMI-based methods in that barcoding can be achieved by a single round of primer extension rather than multiple cycles of PCR. For two-ended labeling, an additional round of primer extension with reverse UMI primers will be done after removing forward UMI primers. The UMI-labeled DNA will be further amplified by universal primers before sequencing.

Any of the methods disclosed herein can further include removal of one or more primers or other components of any previous step before moving to the next step. For example, in some embodiments, the UMI primer(s) is removed after a single cycle of PCR used to add it to the end of a target nucleic acid(s). Thus, in some embodiments, the method include one cycle of PCR with UMI primer(s) followed by removal of the UMI primer(s) prior to amplification of the amplicon with a set of universal and target nucleic acid specific primers (e.g., one-end label methods). In some embodiments, the method include one cycle of PCR with UMI primer(s) followed by removal of the UMI primer(s), followed by prior to one cycle of PCR with reverse UMI primer(s) followed by removal of the UMI primer(s), followed by amplification of the amplicon with a universal primer.

An alternative labeling method that is particularly effective for labeling mtDNA includes one or more of the steps of FIG. 5. For example, a method of labeling mtDNA can including

(i) optional restriction enzyme (e.g., BsrG1) digest of only the nuclear DNA in a nucleic acid sample including nuclear and mitochondrial DNA;

(ii) optionally, additionally, or alternatively treatment of the nucleic acid sample with lambda exonuclease; and

(iii) labeling of the remaining mtDNA with UMI labels, priming sites, and bar codes using EZ-Tn5 transposon.

The method can further include optional amplification of the labeled mtDNA sequence(s) as introduced above, and sequence of the labeled and optionally amplified amplicons as discussed below.

In this method, the restriction enzyme (e.g., BsrG1) is used to digest only the nuclear DNA to short fragments. The digested DNA can be further treated by lambda exonuclease. The circular mtDNA will be protected from two-round digestion. This will enrich mtDNA for being labeled by EZ-Tn5 transposon. After that, UMIs labeled mtDNA can be further enriched and purified by size-selection based method, e.g. Bluepippin or gel extraction. The mtDNA after transposition contains UMIs, priming sites, and barcodes. The primers integrated into the mitochondrial genome permit amplifying only mtDNA. The barcode sequences permit multiplexing samples before final amplification. By pooling samples together, PCR can be carried out with a higher amount of starting material (template), which will improve the PCR performance.

If the transposase-directed method is still not sensitive enough, mtDNA can be first amplified from a single cell. This gives rise to an indiscriminative magnification of all mtDNA in the cell. After that either PCR-directed or transposase-directed method can be used to label mtDNA with UMIs.

C. Methods of Sequencing and Sequence Analysis

The foregoing methods can be tethered to a larger method that includes sequencing. For example, a method of determining the sequence of a target nucleic acid can include:
(i) labeling of one or more target nucleic acids according to a labeling method disclosed herein and optionally amplifying the amplicons;
(ii) sequencing the labeled amplicons;
(iii) optionally grouping sequences having the same UMI into one or more groups, preferably wherein each group consist of a sequence having the same UMI;
(iv) determining the sequence of each target nucleic acid sequence by determining the consensus sequence of each group.

Some embodiments include identifying polymorphisms or other sequence variation in one or more of the target nucleic acids, for example compared to a control sequence or another nucleic acid sample.

In more specific embodiments, the polymorphism is a single nucleotide polymorphism (SNP).

In some embodiments, the sequencing step includes use of long-read sequencing technology, such as for example, using a Nanopore sequencing. Oxford Nanopore sequencing is an emerging third-generation sequencing technology, that can generate ultra-long reads exceeding 800 kb (Jain et al., Nat Biotechnol 36, 338-345, doi:10.1038/nbt.4060 (2018)) in a portable device called MinION. These long-reads come without much compromise on reads consensus accuracy since the sequencing errors are mostly random (Loman et al., Nat Methods 12, 733-U751, doi:10.1038/Nmeth.3444 (2015)). They hold great promise in calling and phasing variants, assembling scaffold, and prospectively detecting epigenetic marks (Cretu et al., Nat Commun 8, 1326, doi:10.1038/s41467-017-01343-4 (2017), Simpson et al., Nat Methods 14, 407-410, doi:10.1038/nmeth.4184 (2017)).

In particular embodiments, the methods include preparing a sequencing library, for example a Nanopore sequencing library such as a 1D ligation library from the labeled amplicons.

Any of the steps can include bioinformatics tools or techniques, and can include bioinformatics analysis. Exemplary preferred analysis include, but are not limited to, basecalling, sequence alignment(s), polymorphism identification and combinations thereof. An exemplary bioinformatics analysis can include, for example, any of the steps in FIG. 3C.

The sequencing error of Nanopore comes mainly from the algorithm used to interpret raw signals, which is the basecalling process. Signal-level algorithm for analyzing variations is not relied on the basecalled reads, but works directly on the raw electronic signal. Results indicate that cwDTW, an algorithm developed for the end-to-end mapping between the raw electrical current signal sequence and the reference genome, can accurately and effectively handle the ultra-long signal sequences of Nanopore sequencing (Han et al., Bioinformatics 34, 722-731, doi:10.1093/bioinformatics/bty555 (2018)). This algorithm can be modified to group reads and detect mutations after single-cell individual mtDNA sequencing. The established SNPs calling pipeline as shown in the Examples (e.g., FIG. 3C) can serve as a benchmark to evaluate the performance of new algorithms

The algorithm typically needs to identify reads with the same UMI and use these to get the consensus sequence of the allele. In some embodiments, this step is done with read-clustering algorithms that work well for fixed-length reads of short-read sequencing (e.g. Illumina). However, this strategy could miss reads with complex changes such as those uncovered by long-read sequencing, which prevents detection of deletions, insertions and complex structural variants. In some embodiments, the data analysis includes a BLAST-like strategy to locate UMI sequence in reads regardless of length and structure. This type of analysis thus preserves the sequence information of all types of alleles and their frequency. An exemplary pipeline is illustrated in FIG. 9B. An algorithm referred to here VAULT carries out this pipeline.

VAULT uses several published algorithms for UMI extraction, alignment, and variant calling. The whole analysis can be done with one command In brief, Nanopore reads are trimmed to remove adapter sequences, and then aligned to the reference gene for extraction of mappable reads. VAULT extracts UMI sequence, followed by counting of the occurrence of each UMI, which reflects the number of reads in each UMI group. If a structured UMI (NNNNTGNNNN (SEQ ID NO:2)) is used in the experiment, the program will also check the UMI structure and separate them to perfect UMIs and wrong UMIs. Next, based on a user-defined threshold of minimum reads per UMI group, the program bins reads for eligible UMIs. The grouped reads will be subjected to alignment, followed by SNP and SV calling. After finishing all variant calling, a final data cleanup is performed to combine individual variant call files (VCF) together and filter the VCF. The number of reads in UMI groups and the corresponding UMI sequence will be written in the ID field of the VCF. Individual folders named after the UMI sequence will be saved to contain the alignment summaries and BAM files of every UMI group. VAULT supports both long-read data and single-end/paired-end short-read data. The data analysis pipeline employs parallel computing for each UMI group, which avoids crosstalk during data analysis and accelerates the process. A typical analysis of 2.5 million long reads will take around four hours on a 32-core workstation. Any of the disclosed methods can include a data analysis step(s) including any one of more steps carried out by VAULT. In some embodiments, the methods include all of the steps carried out by VAULT.

D. Nucleic Acid Samples

The nucleic acid sample can be, for example, nuclear genomic DNA, mitochondrial genomic DNA, or a combination thereof. The sample can be prokaryotic or eukaryotic cells. The cells can be, for example microbial (e.g., bacterial, viral, etc.), or from a higher organism, for example, an animal such as mammal including humans.

The source of the nucleic acid sample can from, for example, any integer between 1 and 1,000,000 cells inclusive, or any range formed of two integers there between, for example, between 1 and 10,000, 1 and 1,000, 1 and 100, 1 and 10, or 1 single cell.

The source of the nucleic acid sample can one single nuclei or one single mitochondrion.

In some embodiments, any of the disclosed methods further include isolating the nucleic acid sample from, for example, a cell or cells. The isolation can include releasing the target nucleic acid sample by, for example, lysing the cell(s). The lysing can be chemical, enzymatic, osmotic, mechanical, or a combination thereof.

In some embodiments, the target nucleic acid is, or is suspected of, being related to aging or an age-related disorder.

Any of the methods can include one or more restriction digestions of the nucleic acid sample prior to the first cycle of PCR.

Any of the methods can include removing contaminants (e.g., one or more of primers, dNTPs, RNA, etc.), before the first cycle of PCR, after the first cycle of PCR, or any second or subsequent cycle of PCR, or any combination thereof.

Any of the disclosed methods can further include amplifying the nucleic acid sample, or a fraction thereof, prior to labeling.

Any of the disclosed methods can further include one or more rounds of enrichment and/or purification of the nucleic acid sample, target nucleic acid, amplicons, or otherwise labeled nucleic acid. The enrichment and/or purification can include size selection.

### III. Exemplary Embodiments

In a proof-of-concept study discussed in more detail below, total DNA was extracted from cells. For samples of limited quantity, cells were resuspended in PBS and lysed in RIPA buffer on ice to release DNA, followed by DNA cleanup with 1X AMPure XP beads. The purified DNA was subjected to PCR-directed labeling using the UMI primers (FIG. 1B).

The UMI primer contained three parts: a universal primer for amplifying the DNA, an UMI structure for labeling individual DNA molecule, and a gene-specific primer for targeted DNA amplification. An exemplary universal sequence is CATCTTACGATTACGCCAACCAC (SEQ ID NO:1). This sequence is designed to avoid forming secondary structure and nonspecific amplification of the human and the mouse genome.

An exemplary UMI sequence is NNNNTGNNNN (SEQ ID NO:2), wherein “N” is any nucleotide (e.g., A, G, T, or C). This sequences is designed to avoid homopolymers.

The gene-specific primers can be any sequences to amplify a gene of interest using PCR.

This strategy can be used to label one or both ends of a gene of interest. An exemplary method for labeling one end of a gene of interest includes using one universal/UMI primer to label one end of the gene of interest according to the following PCR parameters: 98° C. 1 min, 70° C. 5 s, 69° C. 5 s, 68° C. 5 s, 67° C. 5 s, 66° C. 5 s, 65° C. 5 s, 72° C. 5 min (depends on the amplicon length and the polymerase), 4° C. hold.

After that, another universal/UMI primer is optionally used to label the other end of the amplicon, using the same or similar PCR parameters. In some embodiments, resulting amplicon can have a random combination of two different UMI.

The labeled DNA can be purified. In an exemplary protocol, the DNA is purified using 0.8X AMPure XP beads to remove the primers.

Next, the universal primer can be used to amplify all of the labeled DNA for sequencing.

This method can be used to label both linear DNA and circular DNA with UMIs.

Next, the amplified DNA (e.g., the amplicon(s)) is sequenced. Any sequencing platform can be used and selected based on the application. For example, if the amplicon is long, then a long-read sequencing technology such as Oxford Nanopore, Pacific Biosciences can be used to generate reads spanning the whole amplicon with two UMIs.

An exemplary pipepline is described in more detail in the working Examples and Figures associated therewith. The example is illustrated using mitochondrial DNA, but it will be appreciated that any nucleic acid (e.g., a source of DNA, genomic or otherwise), can serve as the source material and can be substituted for mitochondrial DNA in the experiment.

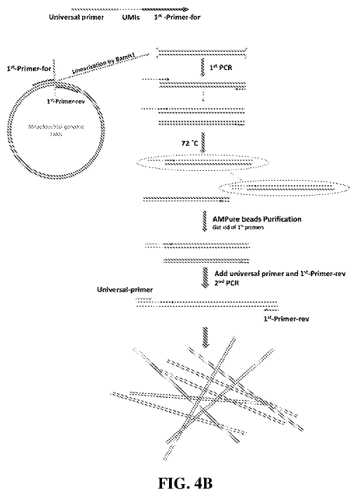

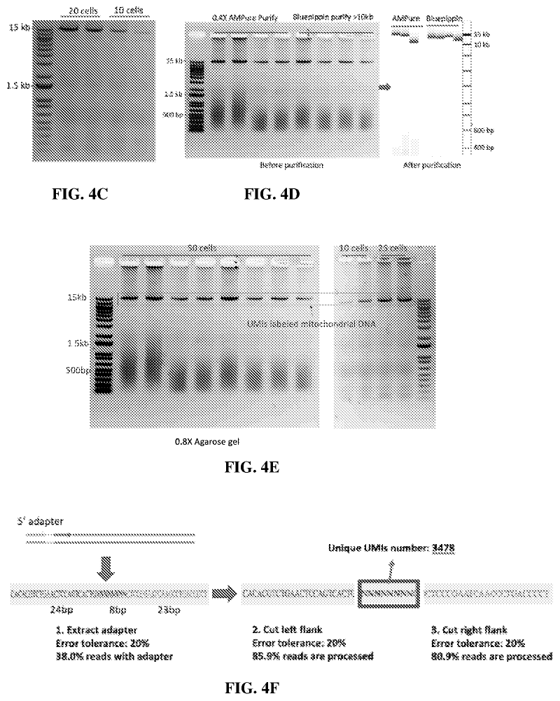

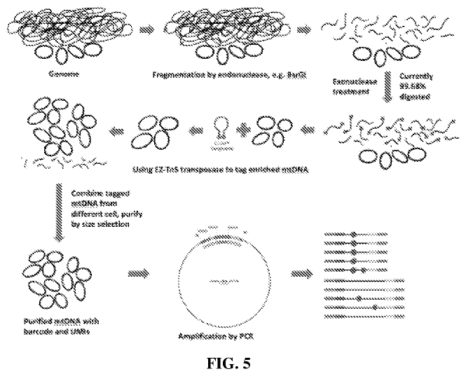

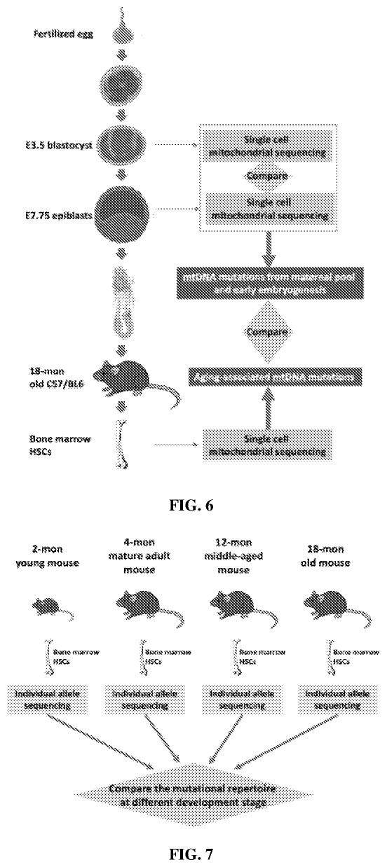

An exemplary pipeline is depicted in FIG. 4A-4B and illustrates labeling mitochondrial DNA in humans for single-cell mitochondrial sequencing. A single cell is sorted by manual pipetting and resuspended in 0.5 μl PBS, followed by lysis in 10 μl RIPA buffer on ice for 15 mins.

After cell lysis, the reaction is diluted with water and the DNA is digested by BamH1 in 50 μl reaction. After that, 0.8X AMPure XP beads are used to clean up the DNA and elute the purified DNA in 10 μl water. The purified DNA is subjected to PCR-directed labeling using primer CATCTTACGATTACGCCAACCACTGNNNTGNNNCTCCCGAATCA ACCCTGACCC (SEQ ID NO:3) and CTATTGGTGCGGGGGCTTTGT (SEQ ID NO:4). The PCR reaction is 11 μl Platinum™ SuperFi™ PCR Master Mix, 1 μl primer mix (final concentration 0.5 μM each), and 10 μl purified DNA. The PCR parameters are 98° C. 1 min, 70° C. 5 s, 69° C. 5 s, 68° C. 5 s, 67° C. 5 s, 66° C. 5 s, 65° C. 5 s, 72° C. 10 min, 4° C. holds.

After UMI labeling, the whole DNA is amplified using the primers 5′CTATTGGTGCGGGGGCTT3′ (SEQ ID NO:5) and 5′CTATTGGTGCGGGGGCTT3′ (SEQ ID NO:5). The amplicon is further purified by 0.8X AMPure XP beads.

Data reported in the Examples below indicate that this protocol can succeed in labeling mitochondrial DNA in as few as 10 cells (FIG. 4C-4E).

These UMI-labeled DNA were used to prepare an Oxford Nanopore 1D ligation library to sequence on a Nanopore MinION sequencer. 3478 unique UMIs were recovered (FIG. 4F). These results show that this method can label small amounts of DNA for downstream analysis of rare genetic variants in human cells.

QIAEX II Gel Extraction Kit with a higher DNA recovery of 80% can be used to purify DNA to increase the yield of, for example, the amplicons. The purified high molecule weight DNA can be used to make, for example, a 1D library using the ligation sequencing kit, and be sequenced on, for example, the R9.4.1 flow cell. The new-released kit and flow cell provide an improved sequencing yield up to 10 GB per flow cell.

Another consideration is the percentage of reads with UMIs. From the sequencing result of one-end labeled mtDNA, it is believed that 41.17% of reads can be grouped by UMIs for calling variants. This estimation can be revised by additional sequencing runs. Samples can be multiplexed to achieve the maximum usage of the flow cell.

These compositions and methods and be used to improve the accuracy and sensitivity of next-generation and third-generation sequencing. They are compatible with most sequencing platforms in the market and therefore holds a great promise to improve the application of genetic testing in clinical diagnosis.

### IV. Applications

The disclosed individual-nucleic acid molecule labeling can improve nuclear and mitochondrial genome analysis from a population of cells. It can provide the information of the individual nuclear allele in a population of cells, and the information of the comprehensive mitochondrial genome within one cell.

In some embodiments, UMI labeling is combined with Oxford Nanopore sequencing technology. By combining the disclosed individual-DNA molecule labeling and long-read Nanopore sequencing technology, new insights into the roles of genomic alteration in aging processes are gained and can facilitatefurther study to improve healthspan and longevity.

In some embodiments, the compositions and methods are used for metagenomic analysis, e.g., analysis bacterial or viral genomes, analysis of hospital or environmental sample, e.g., for selective identification of antibiotic-resistant microbes.

Exemplary applications are discussed in more detail below.

A. Single Cell Analysis of Genomic Variation of Mitochondria

Single-mitochondrion sequencing has been achieved by isolating single mitochondrion in a single cell and subsequently amplifying it to three fragments (Morris et al., Cell Rep 21, 2706-2713, doi:10.1016/j.celrep.2017.11.031 (2017)), but sequencing only one of the thousands of mtDNA within one cell does not lead to a better interpretation of the causality of mtDNA mutation and related phenotype.

The disclosed compositions and methods can be used to label individual mitochondria in a single cell. High-throughput sequencing of the labeled mtDNA can be carried out using long-read Nanopore sequencing. In addition, bioinformatics can be used for signal-level reads manipulation for accurately detecting mitochondrial mutations.

Thus, the compositions and methods can be used to facilitate the discovery of potentially pathogenic mtDNA mutations that lie below the current detection limit, study of the relationship between the levels of heteroplasmy and cellular phenotype, and contribute to a better understanding of mitochondrial mutations and aging.

The preliminary data below shows and individual-DNA labeling method using material from ten 293T cells. 293T cells are derived from a human embryonic kidney and qPCR data showed 293T cells have about 1000 copies of mtDNA.

In another embodiment, mtDNA is labeled in a single oocyte. Published data showed that mouse oocyte has an average 249.4 k mitochondria (Cree et al., Nat Genet 40, 249-254,doi:10.1038/ng.2007.63 (2008)), which is more than 100 times of that in the kidney (D'Erchia et al., Mitochondrion 20, 13-21, doi:10.1016/j.mito.2014.10.005 (2015))

The disclosed compositions and methods can be used to determine if aging-associated mtDNA mutations originated from low-level heteroplasmic mutations during early embryo development or acquired during the adult life. To do so, the mtDNA mutational load is surveyed in a single cell isolated from early embryos and adult stem cells in aged subjects. In some embodiments, the materials is from humans or mice.

It has been shown that mtDNA copy number increases significantly when the replication suddenly accelerates on embryonic day 7.5 (E7.5) in mouse (Cree et al., Nat Genet 40, 249-254,doi:10.1038/ng.2007.63 (2008), Piko et al., Dev Biol 123, 364-374, doi:Doi 10.1016/0012-1606(87)90395-2 (1987)). Therefore, studying the mtDNA mutational load before and after this time allows survey of both the maternal mtDNA mutations and any potential new mutations due to replication error. Timed-pregnant C57BL/6 mice can be used for collecting single cells from E3.5 blastocyst and E7.75 epiblast (Okamura et al., Genes Genet Syst 90, 405-405 (2015)). Tissue can be dissociated into single cells and subjected to a single-cell individual-mtDNA labeling workflow. In an exemplary embodiment, 30 cells per stage can be sequenced in three biological replicates. The rest of the cells can be saved for repeats and validation experiments.

To produce mice with a strictly identical maternal mtDNA genetic background for later aging analysis, embryos used in previous study can be implanted into pseudopregnant surrogate mothers. Live pups can be kept to, for example, 18 months for collecting aged tissues. A previous study reported that the mtDNA mutations cause a blockage during HSC differentiation (Norddahl et al., Cell Stem Cell 8, 499-510, doi:10.1016/j.stem.2011.03.009 (2011)).

Others have observed an increased level of mutations in the mtDNA control region in single HSCs in normal aged C57BL/6 mice using a PCR sequencing method (Yao et al., Hum Mol Genet 16, 286-294, doi:10.1093/hmg/dd1457 (2007)). However, it is unclear how the mutational load of mtDNA leads to HSC senescence. Applying single-cell individual-mtDNA sequencing to HSCs could help answer this question and also clarify the origin of aging-associated mutations. In an exemplary protocol, bone marrow cells (BMCs) can be flushed from the femur cavity by drilling holes at the tips of the femur with a 25 G needle. Red blood cells can be lysed using the ACK lysis buffer. After extensively washing, BMC will be labeled with antibodies against lineage markers, c-kit, Sca-1, mCD34 and mCD135 to FACS sorted for phenotypic HSCs (Lin-Sca-1+c-kit+mCD34-mCD135-). HSCs can be used immediately or cryopreserved for later analysis. In particular embodiments, 30 cells per cell type are sequenced in three biological replicates. The rest of the cells can be saved for repeats and validation experiments.

By comparing the mtDNA mutation repertoire in stem cells of origin and developmental stage, the origin of mutations in aged tissues can be identified and the question how the accumulation of mtDNA mutations contribute to aging can be answered.

To validate the mutations detected, a different haplotype mtDNA from a phylogenetically distant mouse strain (NZB) can be spiked in the library to check the variant calling sensitivity and accuracy. Ultradeep Illumina sequencing and the digital droplet PCR can be used to identify the mutations.

Mitochondria are vital to life. Mutations in mtDNA can cause infertility, multi-systems diseases, stem cell dysfunction and aging. The mechanisms by which mtDNA mutations contribute to these conditions are not well understood, partly due to the limitations of current methods for the detection and quantification of mtDNA mutations. The disclosed compositions and methods can be utilized to improve the sensitivity and accuracy of mtDNA detection and increase the resolution of mtDNA mutational analysis to the single-cell level. The compositions and method allow researchers to address several key open questions in the field, including characterization of a full-range of pathogenic mtDNA mutations that lie below the current detection limit, mechanistic study of the roles of mtDNA mutation in stem cell function and aging, and provision of diagnostic tools for mitochondrial diseases. Other potential applications include sensitive detection of mtDNA mutations in minute samples for forensic testing and using mtDNA mutation signatures for lineage tracing in humans

B. Decoding the Mutational Processes in Stem Cells

The disclosed compositions and methods can be used to study the development of somatic mutations in stem cells, e.g., hematopoietic stem cells (HSCs), and their influence on aging, by sequencing individual alleles from a population of the cells.

For example, the compositions and methods can be used to investigate HSC aging using the Fanconi anemia mouse model. Previous studies demonstrated that mice harboring the Fanca-/- deficiency give rise to a high level of DNA mutations along with a functional decline in HSCs (Walter et al., Nature 520, 549-552, doi:10.1038/nature14131 (2015), Kaschutnig et al., Cell Cycle 14, 2734-2742, doi:10.1080/15384101.2015.1068474 (2015), Parmar et al., Mutation Research/Fundamental and Molecular Mechanisms of Mutagenesis 668, 133-140, doi:10.1016/j.mrfmmm.2009.03.015 (2009)).

A putative mechanism for how deficiencies of Fanconi anemia repair pathway contribute to accelerated nuclear DNA mutations is described in (Walter et al., Nature 520, 549-552, doi:10.1038/nature14131 (2015)). In homeostatic conditions, HSCs reside in a quiescent state within the bone marrow niche. Stress-induced HSC activation initiates the DNA replication and upregulates energy production via oxidative phosphorylation within the mitochondria. This brings a high level of intracellular ROS, which increases the likelihood of the DNA replication fork colliding with repair intermediates, result in the induction of many stalled replication forks. The Fanconi anemia repair pathway can resolve the stalled replication fork by coordinating the regression of the replicative machinery followed by translesion synthesis and homologous recombination repair. This repair pathway is of high fidelity and prevents DNA mutations. However, for some lesions, the replication fork will collapse, resulting in a DNA double-strand break (DSB), which will in turn promote a locus-specific phosphorylation of cH2AX. Inefficient repair of DNA lesions will lead to cell death, or survive with the addition of DNA mutations. The deficiencies of Fanconi anemia repair pathway will favor error-prone repair of stress-induced DNA damage, leading to an accelerated accumulation of nuclear mutations.

The disclosed compositions and methods can be used to sequence and track the dynamic change and load of mutations in aging, e.g., HSC aging. Exemplary genes include, but are not limited to, those in Table 1:

TABLE 1
List of genes to be sequenced.

| Gene type | Gene |
|---|---|
| DNA repair pathway | TP53, P21, XPB, XPD, TTDA |
| Involved in longevity | LMNA, WRN, CSA, CSB, TOR, S6K, IGF1, IIS |
| Frequently mutated in | DNMT3A, TET2, JAK2, ASXL1, SF3B1, |
| HSCs | SRSF2, RUNX1, NRAS, FLT3 |

These genes were chosen based on the following criteria: 1) genes involved in DNA repair pathway, 2) genes found to impact on longevity (Burtner & Kennedy, Nat Rev Mol Cell Biol 11, 567-578, doi:10.1038/nrm2944 (2010)), 3) genes frequently mutated in HSCs (Moehrle & Geiger, Exp Hematol 44, 895-901, doi:10.1016/j.exphem.2016.06.253 (2016), Smith & Sudbery, Genome Res 27, 491-499, doi:10.1101/gr.209601.116 (2017)) (Table 1).

Thus, the compositions and method disclosed herein can be used to investigate how somatic mutations accumulate in the earliest stage of stem cell aging, e.g., HSC aging, and the relationship between mutational load and stem cell, e.g., HSC, senescence. The result may unveil new ways of slowing the aging and extending the healthy lifespan.

The highly sensitive and accurate detection of rare mutations in a population of cells can be achieved by combining the individual-DNA molecule labeling method (FIG. 1C), the long-read Nanopore sequencing technology, and the signal-level data-analysis algorithm. The sensitivity of the method can be determined using an artificial “rare mutations” sample by pooling different haplotype of DNA together at a series of ratio, for example, gene edited cell lines with single nucleotide change and gene deletion.

Genomes from wild-type and gene edited cell lines can be extracted using QIAGEN DNeasy blood and tissue kits. Two genomes can be pooled at 1:1000, 1:10000, 1:100000, which equals to 0.1%, 0.01%, and 0.001% allele frequency, respectively. The individual-DNA molecule labeling method can be used to label individual alleles in the mixed genome. A 1D library can be prepared and sequenced on Nanopore MinION. Signal-level algorithm of data analysis can be used to group reads based on UMIs and call variants. In some embodiments, the sequence coverage is 200X per grouped reads. Ultra-deep Illumina sequencing of the same samples can be done as a reference.

The frequency of HSCs in bone marrow is about 0.01% of total nucleated cells and about 5000 can be isolated from an individual mouse depending on the age, sex, and strain of mice as well as purification scheme utilized (Challen et al., Cytometry A 75, 14-24, doi:10.1002/cyto.a.20674 (2009)). This means a sensitivity of 0.01% of allele frequency will be enough to detect one allele mutation in 5000 cells. It is believed to be difficult to detect rare mutations with less than 1% allele frequency use Illumina sequencing because of its intrinsic sequencing error (Shendure & Ji, Nature Biotechnology 26, 1135-1145, doi:10.1038/nbt1486 (2008)). The disclosed method is believed to be able to exceed this sensitivity. If the mutations can be called at 0.001% allele frequency, a smaller allele frequency of samples can be used to detect the sensitivity of this method.

The disclosed workflow can also be used to survey the mutational processes in HSC aging in mouse model of Fanca-/- deficiency (FIG. 7). Previous studies showed that Fanca-/- mouse appeared normal, without clear congenital malformations or growth retardation (Cheng et al., Human Molecular Genetics 9, 1805-1811, doi:DOI 10.1093/hmg/9.12.1805 (2000)), which make it possible to study the aspect of HSC aging. This mouse strain has a 5-fold higher level of DNA mutations in HSCs and a relatively normal number of progenitor bone marrow cells (Walter et al., Nature 520, 549-552, doi:10.1038/nature14131 (2015), Kaschutnig et al., Cell Cycle 14, 2734-2742, doi:10.1080/15384101.2015.1068474 (2015), Sperling et al., Nat Rev Cancer 17, 5-19, doi:10.1038/nrc.2016.112 (2017)). The impaired DNA damage (e.g. double-strand break) repair pathway by Fanca-/- deficiency gives rise to an accumulation of mutations, including single nucleotide variants, deletions, insertions, and translocations (Palovcak et al., Cell Biosci 7, 8, doi:10.1186/s13578-016-0134-2 (2017)). And the proportion of mutations could be very low in the whole HSCs population. The full spectrum of mutations, especially rare mutations and structural variants, is hard to be detected by short-reads Illumina sequencing.

Individual-DNA molecule labeling method together with long-range PCR (up to 16.5 kb) and long-reads Nanopore sequencing can solve this problem. The well-studied hematopoietic system allows for focus on several key genes with high mutational frequency (Moehrle & Geiger, Exp Hematol 44, 895-901, doi:10.1016/j.exphem.2016.06.253 (2016)). Bone marrow cells (BMCs) can be flushed from the femur cavity by drilling holes at the tips of the femur with a 25 G needle. Red blood cells can be lysed using the ACK lysis buffer. After extensively washing, BMC can be labeled with antibodies against lineage markers, c-kit, Sca-1, mCD34 and mCD135 to FACS sorted for phenotypic HSCs (Lin-Sca-l+c-kit+mCD34-mCD135-). HSCs can be either used immediately or cryopreserved for later analysis. In an exemplary embodiment as assay include sequence of the UMIs labeled amplicon of 22 genes in Table 1 using three mice per age (2 months, 4 months, 12 months, 18 months) in three biological replicates. 5000 HSCs can be isolated from each mouse and the cells lysed in RIPA buffer followed by DNA purification by AMPure beads. This DNA extraction method has been shown to work well in small numbers of cells in experiments described below (FIGS. 4A-4E). After that, the extracted DNA can be subjected to the workflow described herein to detect mutations in these genes. To validate detected mutations, the mutated DNA can be cloned into a plasmid and sequenced by Sanger sequencing. The digital droplet qPCR can be used to confirm the mutations. By comparing the mutation repertoire in HSCs at different developmental stage; 1) The patterns of mutational expansion in the early stage of HSCs senescence; 2) The unique mutational constitution and pattern at each stage of HSC aging; and 3) A more accurate mutational load in HSC aging, can be investigated.

It is well known that genomic instability contributes to aging. However, there is still a fundamental gap in understanding the very beginning stage of aging senescence which is how the cellular mutations get accumulated in cells. The disclosed compositions and methods can be used to address this question and lead to a better understanding of genomic mutations and HSC aging. Besides its impact on aging research, the technology can make possible DNA sequencing in allele-level sensitivity on various topics and applications (such as detection of minimal residual disease). Exemplary use such as those described herein can provide new insights into the roles of genomic alteration in aging processes and facilitate further study to improve healthspan and longevity.

Besides its influence on aging research, the disclosed compositions and methods can be used for range of other application. For example, DNA sequencing in allele-level sensitivity on various topics and applications: such as detection of minimal residual disease), single cell mitochondrial sequencing can be used for diagnosing mitochondria-related diseases, bacteria-specific gene sequencing to identify the bacterial strains, and ultra-sensitive detection of rare genetic variant in biological samples (e.g. forensic test).

The disclosed compositions and methods of use thereof can be further understood through the following numbered paragraphs.
1. A unique molecular identifier (UMI) primer comprising a universal primer sequence, a unique molecular identifier (UMI) sequence, and a first target nucleic acid binding sequence.
2. The primer of paragraph 1 wherein the orientation of the universal primer sequence, unique molecular identifier (UMI) sequence, and first target nucleic acid binding sequence is 5′ universal primer sequence, unique molecular identifier (UMI) sequence, first target nucleic acid binding sequence 3′.
3. The primer of paragraphs 1 or 2 wherein the universal primer sequence comprises the sequence CATCTTACGATTACGCCAACCAC (SEQ ID NO:1), the reverse sequence thereof, the complementary sequence thereto, the reverse complementary sequence thereof.
4. The primer of any one of paragraphs 1-3, wherein the UMI sequence comprises a random sequence (such as NNNN or NNNNNNN), a partially degenerate nucleotide sequence (such as NNNRNYN or NNNNTGNNNN (SEQ ID NO:2), wherein “N” can be A, T, G, or C, “R” can be G or A, and “Y” can be T or C, or the reverse sequence thereof, the complementary sequence thereto, or the reverse complementary sequence thereof, optionally wherein the UMI sequence is between about 5 and about 100 nucleotides in length.
5. The primer of any one of paragraphs 1-4, wherein the first target nucleic acid binding sequence binds at or near or a gene of interest.
6. The primer of paragraph 5, wherein the first target nucleic acid binding sequence binds to mitochondrial DNA.

7. The primer of any one of paragraphs 1-6 comprising

(SEQ ID NO: 3)
CATCTTACGATTACGCCAACCACTGNNNTGNNNCTCCCGAATCAA
CCCTGACCC

8. A method of labeling a target nucleic acid comprising carrying out at least one cycle of polymerase chain reaction using a first primer of any of paragraphs 1-7 and a nucleic acid sample comprising a nucleic acid sequence to which the first target nucleic acid binding sequence of the primer can bind.
9. The method of paragraph 8 wherein the first cycle of PCR further comprises a second primer comprising a second target nucleic acid binding sequence and the target nucleic acid comprises a nucleic acid sequence to which the second target nucleic acid binding sequence of the second primer can bind.
10. The method of paragraphs 8 or 9, wherein a second and optionally one or more subsequent cycles of PCR further comprises a second primer alone or in combination with the first primer, the second primer comprising a second target nucleic acid binding sequence, and the target nucleic acid comprising a nucleic acid sequence to which the second target nucleic acid binding sequence of the second primer can bind.

11. The method of paragraphs 9 or 10, wherein the second primer further comprises the same or a different universal primer sequence as the first primer, or the reverse sequence thereof, the complementary sequence thereto, or the reverse complementary sequence thereof.
12. The method of any one of paragraphs 9-11, wherein the second primer further comprises the same or different UMI as the first primer, or the reverse sequence thereof, the complementary sequence thereto, or the reverse complementary sequence thereof.
13. The method of any one of paragraphs 9-12 wherein the orientation of the universal primer sequence, unique molecular identifier (UMI) sequence, and second target nucleic acid binding sequence of the second primer is 5′ universal primer sequence, unique molecular identifier (UMI) sequence, second target nucleic acid binding sequence 3′.
14. The method of any one of paragraphs 9-13 comprising any integer between 1 and 100 inclusive subsequent cycles of PCR.
15. The method of any one of paragraphs 8-14, wherein the nucleic acid sample is nuclear genomic DNA, mitochondrial genomic DNA, or a combination thereof.
16. The method of any one of paragraphs 8-15, wherein the source of the nucleic acid sample is any integer between 1 and 1,000,000 cells inclusive, or any range formed of two integers there between, for example, between 1 and 10,000, 1 and 1,000, 1 and 100, 1 and 10, or 1 single cell.
17. The method of any one of paragraphs 8-16, wherein the source of the nucleic acid sample is one single nuclei or one single mitochondrion.
18. The method of any one of paragraphs 8-17, wherein the nucleic acid sample is isolated from a cell or cells.
19. The method of paragraph 18, wherein the isolation comprises releasing the target nucleic acid sample by lysing the cell(s).
20. The method of any one of paragraphs 8-19, wherein the nucleic acid sample is subjected to a restriction digestion prior to the first cycle of PCR.
21. The method of any one paragraphs 8-20 further comprising removing contaminants (e.g., one or more of primers, dNTPs, RNA, etc.), before the first cycle of PCR, after the first cycle of PCR, after the last cycle of PCR, or any combination thereof.
22. The method of any one of paragraphs 8-21 further comprising any integer between 1 and 100 inclusive cycles of PCR comprising primers that bind to the one or more universal primer sequences.
23. The method of any one of paragraphs 8-22 comprising two or more first and second primer sets, each first and second primer set comprising different target nucleic acid binding sequences designed to label and optionally amplify different target nucleic acids.
24. The method of paragraph 23, wherein the UMI sequence for each first primer of each primer set is the same.
25. The method of paragraph 23, wherein the UMI sequence for each first primer of each primer set is different.
26. The method of any one of paragraphs 23-25, wherein the UMI sequence for each second primer of each primer set is the same.
27. The method of any one of paragraphs 23-25, wherein the UMI sequence for each second primer of each primer set is different.
28. A method of determining the sequence of a target nucleic acid comprising
   (i) labeling one or more target nucleic acids according to the method of any one of paragraphs 8-27;
   (ii) sequencing the labeled amplicons;
   (iii) optionally grouping sequences having the same UMI into one of more groups;
   (iv) determining the sequence of each target nucleic acid sequence by determining the consensus sequence of each group.
29. The method of paragraph 28 further comprising (v) identifying polymorphisms in one or more of the target nucleic acids.
30. The method of paragraph 29, wherein the polymorphism is a single nucleotide polymorphism (SNP).
31. The method of any one of paragraphs 28-30, wherein the sequence comprises long-read sequencing technology.
32. The method of paragraph 31, wherein the long-read sequencing technology is comprises Nanopore MinION sequencer.
33. The method of paragraph 32, wherein the long-read sequencing technology comprises preparing a 1D ligation library from the labeled amplicons.
34. The method of any one of paragraphs 28-33, wherein any of steps (iii)-(v) are carried out using bioinformatics analysis.
35. The method of paragraph 34, wherein the bioinformatics analysis comprises basecalling, sequence alignment(s), polymorphism identification or a combination thereof.
36. The method of paragraphs 34 or 35, wherein the bioinformatics analysis comprises one or more of steps of FIG. 3C.
37. A method of labeling a target nucleic acid and optionally sequencing the labeled target nucleic comprising one or more of the steps of any of FIGS. 1A, 1B, 1C, 3C 4A, 4B, 4F, or 5.
38. A method of labeling a target nucleic acid and optionally sequencing the labeled target nucleic comprising
   (i) restriction enzyme (e.g., BsrG1) digest of only the nuclear DNA in a nucleic acid sample comprising nuclear and mitochondrial DNA;
   (ii) treatment of the nucleic acid sample with lambda exonuclease;
   (iii) labeling of the remaining mtDNA with UMI labels, priming sites, and bar codes using EZ-Tn5 transposon;
   (iv) sequencing the labeled mtDNA.
39. The method or any one of paragraphs 8-38 further comprising amplifying the nucleic acid sample, or a fraction thereof, prior to labeling.
40. The method of any one of paragraphs 8-39 further comprising one or more rounds of enrichment and/or purification of the nucleic acid sample, target nucleic acid, amplicons, or otherwise labeled nucleic acid.
41. The method of paragraph 40, wherein the enrichment and/or purification comprises size selection.
42. The method of any one of paragraphs 8-41, wherein the target nucleic acid is, or is suspected of, being related to aging or an age-related disorder.
43. A method of one-end UMI labeling comprising a single round of extension of a UMI primer comprising a universal primer sequence, unique molecular identifier sequence, and target nucleic acid binding sequence that hybridizes to a target nucleic acid sequence and optionally removing the UMI primer from the reaction mixture.
44. A method of two-end UMI labeling comprising a single round of extension of a forward UMI primer comprising a universal primer sequence, unique molecular identifier sequence, and target nucleic acid binding sequence that hybridizes to a target nucleic acid sequence and optionally removing the forward UMI primer from the reaction mixture, and a single round of extension of a reverse UMI primer comprising a universal primer sequence, unique molecular identifier sequence, and target nucleic acid binding sequence that hybridizes to a target nucleic acid sequence and optionally removing the reverse UMI primer from the reaction mixture.
45. The method of paragraphs 43 or 44, further comprising amplifying the one-end or two-end labeled target nucleic acids by PCR with a universal primer alone or in combination with a target nucleic acid specific primer, wherein the cycles of PCR amplify the one- or two-end UMI labeled target nucleic acid.
46. A method of determining the sequence of a target nucleic acid comprising
   (i) labeling one or more target nucleic acids according to the method of any one of paragraphs 43-45;
   (ii) sequencing the labeled amplicons;
   (iii) optionally grouping sequences having the same UMI into one of more groups;
   (iv) determining the sequence of each target nucleic acid sequence by determining the consensus sequence of each group.
47. A method of labeling a target nucleic acid and optionally sequencing and optionally analyzing the labeled target nucleic comprising one or more of the steps of any of FIGS. 8, 9A, 9B, 12A, or any combination thereof.

### EXAMPLES

### Example 1: Development of a Method for Labeling Individual DNA Molecules

Methods

A PCR-directed method has been developed to label individual DNA molecules in cells. The unique molecular identifiers (UMIs) are used to correct the errors during PCR (Smith & Sudbery, Genome Res 27, 491-499, doi:10.1101/gr.209601.116 (2017)). (FIG. 1A). In general, DNA is amplified by two rounds of one-cycle PCR with respective UMI-containing primers. After that, two universal primers are used to amplify the labeled amplicons (FIG. 1C). In the end, the labeled DNA come from different samples are pooled together to make a library that can be sequenced on a Nanopore MinION device.

The universal primers are designed to avoid non-specific amplification in either the human or mouse genome (FIG. 1E). The UMIs structure is designed to avoid secondary structure. Because this is a PCR based method, it is applicable to label any DNA in the cell.

Different polymerases were tested in the PCR reaction to efficiently amplify the 16.5 kb of full-length mtDNA (FIG. 1D). The whole genome extracted from 293T cells by QIAGEN DNeasy Blood & Tissue Kits are used to label the 16.5 kb of full-length mtDNA.

Results

Results showed that full-length mtDNA could be labeled with UMIs using this pipeline (FIG. 1E).

### Example 2: Establishment of Nanopore MinION Sequencing Platform

Materials and Methods

To test the performance of Nanopore MinION sequencer in the Stem Cell and Regeneration lab, several trial sequencing runs were done on R9.4 and R9.5 flow cells with Rapid, 1D and 1D2 library preparation kits.

Results

The rapid and 1D kits are compatible with R9.4 flow cells to provide standard 1D reads (sequence one strand of input DNA), while the 1D2 kit is compatible with R9.5 flow cells to generate a mix of 1D reads and 1D2 reads (sequence one strand followed by its complementary strand). In general, the 1D and 1D2 kits provide the best yield and alignment identity of raw reads. A 24 h sequencing run using the 1D library preparation kit on a R9.4 flow cell can generate 1.4 GB of reads, while 48 hours of sequencing run using the 1D2 kit on a R9.5 flow cell can generate about 1.9 GB of reads (Table 2).

TABLE 2
Summary of trial sequencing run using different Nanopore kits

| | Library | Running | Reads | Average |
|---|---|---|---|---|
| | preparation | time | yield | reads |
| DNA | kits | (hours) | (GB) | length (bp) |
| Mouse | Rapid | 48 | 0.7 | 2300 |
| mitochondria | 1D | 24 | 1.5 | 5859 |
| Amplicon | | | | |
| E. coli K12 | Rapid | 24 | 0.6 | 9363 |
| genome | 1D² | 48 | 1.9 | 5353 |
| | 1D | 4 | 0.3 | 8055 |

The alignment of reads to the reference genome showed an even coverage, which means the labeling and sequencing method comes without regional bias (FIG. 2A). In particular, reads are generated at the same time of sequencing run, which allows real-time control of reads quality and yield. Combining with the feature that flow cells can be washed and reused, this real-time control could potentially reduce the sequencing cost.

The rapid kit uses a transposase-based method to add sequencing adapters, which will fragment DNA and make it not suitable for amplicon sequencing. But it is good for whole genome sequencing since it does not ask for the fragmented genomic DNA. 1D and 1D2 kits use a ligation-based method to add sequencing adapter so that they are suitable for the disclosed application. The 1D2 reads show a higher consensus accuracy, but it takes more time to prepare library and the additional procedure lead to the shearing of DNA.

E. coli genome sequencing showed that 1D kit can generate a higher average length of reads compared with 1D2 kit (Table 2). Based on this, the 1D kit was selected for sequencing amplicon after individual-DNA molecule labeling.

Next, the ability of Nanopore sequencing by the 1D kit to generate reads spanning the full length of the amplicon was tested Amplicons of different length, including 7.7 kb, 8.6 kb, 11 kb, 11.9 kb, 12.7 kb, and 16.5 kb, were tested. The length distribution showed that even 16.5 kb amplicons can be sequenced in a single read (FIG. 2B-2D).

In addition, a number of small fragments appeared in the reads of some sequencing runs. Further analysis showed those reads cannot be mapped to the reference. It is believed that they come from the random priming of primers in long-range PCR and can not be purified by the size selection of AMPure beads. This indicates that an additional size selection either by gel extraction or Bluepippin is preferred.

### Example 3: Establishment of an Exemplary Bioinformatics Pipeline to Analyze Long-Read Data

Materials and Methods

Nanopore sequencing is known to generate ultra-long reads which are much longer than any other sequencing platform in the market. Those reads are error prone with an average alignment identity of 82.73% (Jain et al., Nat Biotechnol 36, 338-345, doi:10.1038/nbt.4060 (2018)). An exemplary bioinformatic pipeline using published algorithms for a proof-of-principle study.

Several of prevalent algorithms were tested to determine the performance of alignment and SNPs calling, including bwa-mean v0.7.17, minimap2.1, graphmap v0.5.2, samtools v1.9, nanopolish v0.IL0 (Jain et al., Nat Biotechnol 36, 338-345, doi:10.1038/nbt.4060 (2018), Li, Bioinformatics 34, 3094-3100, doi:10.1093/bioinformatics/btyl91 (2018), Sovic et al., Nat Commun 7, 11307, doi:10.1038/ncomms11307 (2016)).

The reads in this Lest come from a multiplexed amplicon (8.6 kb and 7.7 kb) sequencing of mouse mtDNA, basecalled by the official algorithm termed Albacore.

Results

Results showed that graphmap has the best performance among the three alignment algorithms, with 89.81% of median alignment identity and 93.40% of base mapped to the reference (FIG. 3A). The alignment shows a comparable amount of errors of mismatches, insertions and deletions. Bwa-mem struggled in phasing those long reads (FIG. 3A).

Targeted sequencing of human HBB locus with distinct coverage distribution was performed to determine if reliable SNPs calling is accessible for Nanopore reads. Sanger sequencing identified that there are the only 3 SNPs located in this gene. Targeted locus amplification was used to enrich this locus and gave rise to uneven coverage after sequencing (de Vree et al., Nat Biotechnol 32, 1019-1025, doi:10.1038/nbt.2959 (2014)) (FIG. 3B). Samtools and nanopolish were used to call the SNPs individually using the default parameters.

Results showed that samtools successfully called the only three SNPs in the BBB gene without any false positive, and the quality score are 222, 217 and 41, respectively. Nanopolish called the three SNPs together with ten false positives, those false positive SNPs come with relatively high-quality score, which makes it hard to filter the SNPs after initially SNP calling. Taken all together, an exemplary bioinformatic pipeline to analyze Nanopore data by using graphmap and samtools was established (FIG. 3C). This pipeline can also be utilized with other signal-level algorithms

### Example 4: mtDNA Labeling in One Hundred 293T Cells

Materials and Methods

The single molecule labeling method was first tested on mitochondrial DNA with a less developed version (only labeling one end of DNA with a UMI). A complete protocol to work from cell lysis to DNA labeling was also developed (FIG. 4A).

Cells are prepared in PBS, and then lysed in RIPA buffer on ice to release mtDNA. After the reaction is diluted and the DNA digested with restriction enzyme to linearize mtDNA.

An AMPure beads-based size selection is performed to clean up DNA and remove small fragments for downstream PCR. One-cycle PCR as described above is used to label mtDNA with UMIs.

The labeled DNA is amplified using universal primers.

A second round of PCR can be done if the yield is not enough for preparing sequencing library.

Currently, this protocol is effective for labeling mtDNA from as few as ten 293T cells (FIG. 4C).

Results

In this Example, the protocol was used to label mtDNA in one hundred 293T cells with UMIs. After collecting enough UMllabeled DNA, DNA was purified by Bluepippin and AMPure beads. The results showed the size selection by Bluepipin can remove all of the small fragments, while size selection by AMPure beads cannot eliminate them (FIG. 4D). DNA from the two purifications were combined and made into a 1D library for sequencing in MinION. A 12-hour of sequencing run generated 407 MB of reads.

A further pipeline was established to extract UMIs and group reads (FIG. 4E). First, the 5′ end sequence of reads was extracted based on the designed adapter structure. A 20% error tolerance is set to cope with the low raw-read accuracy of Nanopore sequencing. 38% of reads are processed. After that, the two flanking sequences were trimmed off the adapter using the same error tolerance, leaving the potential UMIs for downstream analysis.

3478 unique UMIs were identified, and the initial reads can be grouped together based on the UMI sequence.

In summary, the preliminary data show that: (1) The PCR-directed method for individual DNA molecule labeling is feasible to label DNA either from the extracted genome (for nuclear DNA labeling) or from 10 cells (for mtDNA labeling). (2) Nanopore MinION sequencing is capable of sequencing the whole amplicon in one read without bias or compromise in yield. (3) It is possible to use only the long reads produced by Nanopore sequencing to call DNA mutations, even in low-coverage regions.

### Example 5: Long-read Individual-molecule Sequencing Reveals CRISPR-induced Heterogeneity in Human ESCs

Materials and Methods

### Generation of the Knock-In hESC Line

The H1 hESC line was purchased from WiCell and cultured in Essential 8TM medium (ThermoFisher) on hLaminin521 (ThermoFisher) coated plate in a humidified incubator set at 37° C. and 5% CO2.

Electroporation of CAS9 RNP was done using a Neon Transfection System (ThermoFisher) using the following setting: 1600 v/10 ms /3 pulses for 200,000 cells in Buffer R (Neon Transfection kit) premixed with 50 pmol Cas9 protein (CAT#M0646T, New England Biolabs), 50 pmol single guide RNA (sgRNA) and 30 pmol single-stranded oligodeoxynucleotides (ssODN, purchased from Integrated DNA Technologies, Inc.) template.

After 48 hours, single cells were collected and seeded at 1,000 single cells per well (6-well format). Seven days later, single colonies were picked for passaging and genotyping. The EPOR sgRNA sequence including protospacer adjacent motif (PAM) is 5′GCTCCCAGCTCTTGCGTCCA-TGG(PAM)3′ (SE QID NO:8), which was synthesized in vitro by MEGAshortscript™ T7 Transcription Kit (ThermoFisher).
CRISPR-Cas9 Editing of hESCs

CRISPR-Cas9 editing of the PANX1 locus in H1 hESCs were performed in the same way as the generation of knock-in hESCs except for the omission of the ssODN template. After 48 hours, cells are collected for the genome extraction and library preparation. The Pant sgRNA sequence is 5′ATCCGAGAACACGTACTCCG-TGG(PAM)3′ (SEQ ID NO:9) and Pan3 sgRNA is 5′GCTGCGAAACGCCAGAACAG-CGG(PAM)3′ (SEQ ID NO:10).

### UMI Primer Design

The UMI primer contains a 3′ gene-specific sequence, a UMI sequence, and a 5′ universal primer sequence. The 3′ gene-specific sequence is designed with the same principle as PCR primers. A sequence with an annealing temperature higher than 65° C. was chosen to improve specificity to the target gene. The internal UMI sequence consists of multiple random bases (denoted by Ns). The number of random bases is determined by the number of targeted molecules. A short UMI sequence (10-12 nt) was chosen to reduce the sequencing errors within the UMI. A unique sequence structure in the UMI (e.g. NNNNTGNNNN (SEQ ID NO:2)) was chosen to avoid homopolymers that may introduce errors due to polymerase slippage or low accuracy of Nanopore sequencing in these sequences. Several studies have also pointed out that both Illumina and PacBio are prone to errors in such regions (Minoche et al., Genome Biol 12, (2011), Weirather et al., F1000Res 6, 100 (2017)). The structured UMI design also serves as a quality control in the UMI analysis. The 5′ universal primer sequence is used to uniformly amplify all UMI tagged DNA molecules. It is designed to avoid non-specific priming in the target genome.
UMI Labeling

The primers used in this study are shown in Table 3.

TABLE 3
Primers used in this Study.

| Primer | Sequence |
|---|---|
| Universal primer | CATCTTACGATTACGCCAACCAC |
| EPOR UMI primer | CATCTTACGATTACGCCAACCACNNNNN |
| | NNNNNGTTGAGATGCCAGAGTCAGATAC |
| EPOR short revers | TGCCAGCTTTGAGTACACTATC |
| EPOR long reverse | TAACCTCCCGGACCCCAAGTTCG |
| Pan1 UMI primer | CATCTTACGATTACGCCAACCACTGCGGNNNN |
| | NTGNNNNNGACACATTCTCCCAGGCCCTACTT |
| Pan1 reverse | CAGAGTCCCTTCTGCTCTCTGTCC |
| Pan3 UMI primer | CATCTTACGATTACGCCAACCACTGCGGN |
| | NNNNTGNNNNNGCATCCCAGGCCTAATGTGGA |
| Pan3 reverse | GTTGTCAGATTTCCCCACTGGGCTCTT |

Table 3 sequence identifiers: SEQ ID NO:1 (universal primer), SEQ ID NO:11 (EPOR UMI primer), SEQ ID NO:12 (EPOR short reverse), SEQ ID NO:13 (EPOR long reverse), SEQ ID NO:14 (Pani UMI primer), SEQ ID NO:15 (Pani reverse), SEQ ID NO:16 (Pan3 UMI primer), SEQ ID NO:17 (Pan3 reverse).

Genomic DNA is extracted using the Qiagen DNeasy Blood & Tissue Kit. The concentration is determined using a Qubit 4 Fluorometer (ThermoFisher). The UMI labeling step is done by one round of primer extension with a high-fidelity DNA polymerase. The reaction setup is similar to a standard PCR reaction, but with only one UMI primer. The UMI labeling reaction is set up as follows: 50 ng DNA, 1 μM UMI primer, 12.5 μl 2X Platinum™ SuperFi™ PCR Master Mix, and H₂O in a total volume of 25 μl. The UMI labeling is performed on a thermocycler with a ramp rate of 1° C. per second using the following program: 98° C. 1 min, 70° C. 5 s, 69° C. 5 s, 68° C. 5 s, 67° C. 5 s, 66° C. 5 s, 65° C. 5 s, 72° C. (5 min for the 7 kb targets, 10 s for the 168 bp target), 4° C. hold. After UMI labeling DNA is purified by AMPure XP beads, followed by PCR amplification using the universal primer and the gene-specific reverse primer. This amplification will generate enough UMI-labeled DNA for downstream sequencing. In addition to one-ended labeling, two-ended UMI labeling can also be achieved by performing an additional UMI-labeling step with a reverse primer tagged with a UMI (FIG. 9A). Two-ended UMI labeling could increase analyzable reads and provides extra benefit in accuracy. However, because it was found that UMI labeling is limited by primer efficiency, one-ended labeling is believed to cover more molecules. Additional UMI-labeling and purification steps result in higher loss of DNA of interest. Since the procedure of one-ended labeling is simple and efficient, one-end UMI labeling for all experiments in Examples 5 and 6.
Library Preparation and Sequencing

For Nanopore sequencing, library preparations were done using the ligation sequencing kit (Cat# SQKLSK109, Oxford Nanopiore Technologies). The sequencing runs were performed on an Oxford Nanopore MinION sequencer using R9.4.1 flow cells. Base calling of Nanopore reads was done using the official tool termed Guppy (v3.2.1). For PacBio sequencing, library preparations were done using the Sequel Sequencing Kit 3.0. The sequencing runs were performed by the BIOPIC core facility at Peking university (Beijing, China) on a PacBio Sequel using Sequel SMRT Cell 1M v3. HiFi Reads were generated by the official tool termed ccs (v3.4.1). All procedures were preformed according to manufacturer's protocols. For Illumina sequencing, library preparations were performed using the NEBNext Ultra II DNA Library Prep Kit for Illumina. An unrelated RNA library prepared using the same kit was pooled to increase the complexsity of final library. The sequencing of paired-end 150 bp reads was done on an Illumina Miniseq.
Data Processing

VAULT was developed for data analysis. Most of the codes were written in Python 3.7, while some modules were written in Bash. In general, VAULT uses several published algorithms for UMI extraction, alignment, and variant calling. By default, it utilizes cutadapt (Martin, Cutadapt removes adapter sequences from high-throughput sequencing reads. 2011 17, Mar. (2011)), minimap2 (Li, Bioinformatics 34, 3094-3100 (2018)), samtools (Li et al., Bioinformatics 25, 2078-2079 (2009)), and sniffles (Sedlazeck et al., Nat Methods 15, 461-468 (2018)). The whole analysis can be done with one command. In brief, Nanopore reads are trimmed to remove adapter sequences, and then aligned to the reference gene for extraction of mappable reads. Cutadapt is used to extract UMI sequence, followed by counting of the occurrence of each UMI, which reflects the number of reads in each UMI group. If a structured UMI (NNNNTGNNNN (SEQ ID NO:2)) is used in the experiment, the program will also check the UMI structure and separate them to perfect UMIs and wrong UMIs. Next, based on a user-defined threshold of minimum reads per UMI group, the program bins reads for eligible UMIs. The grouped reads will be subjected to minimap2 for alignment, followed by SNP calling by samtools and SV calling by sniffles. After finishing all variant calling, a final data cleanup is performed to combine individual variant call files (VCF) together and filter the VCF. The number of reads in UMI groups and the corresponding UMI sequence will be written in the ID field of the VCF. Individual folders named after the UMI sequence will be saved to contain the alignment summaries and BAM files of every UMI group. VAULT supports both long-read data and single-end/paired-end short-read data. The data analysis pipeline employs parallel computing for each UMI group, which avoids crosstalk during data analysis and accelerates the process. A typical analysis of 2.5 million long reads will take around four hours on a 32-core workstation.

### Results

Molecular consensus sequencing has been developed to enhance the accuracy of next-generation sequencing (NGS) using unique molecular identifier (UMI)(Kinde et al., Proc Natl Acad Sci U S A 108, 9530-9535 (2011), Hiatt et al., Nat Methods 7, 119-122 (2010), Salk et al., Nat Rev Genet 19, 269-285 (2018)). The main concepts of this strategy include barcoding each molecule before amplification, and correcting sequencing error using the consensus sequence of reads containing the same barcode, and hence eliminating the random errors introduced by sequencing chemistry or detection. However, current strategies are inadequate for many types of sequences especially the large structural variants or repetitive sequences (FIG. 8). Single molecule sequencing technologies can better resolve complex genetic variants by providing long reads, but they have a lower raw read accuracy (Salk et al., Nat Rev Genet 19, 269-285 (2018)).

To overcome these limitations, a treated termed targeted Individual DNA Molecule sequencing (IDMseq) was developed. IDMseq ensures that each original DNA molecule is uniquely represented by one UMI group (a set of reads sharing the same UMI) after sequencing, thus preventing false UMI groups and allowing quantification of allele frequency in the original population (FIGS. 8 & 9A). It is designed to be adaptable to various sequencing platforms, and combines error correction by molecular consensus with long-read sequencing, thus enabling sensitive detection of all classes of genetic variants, including SNVs, indels, large deletions, and complex rearrangements.

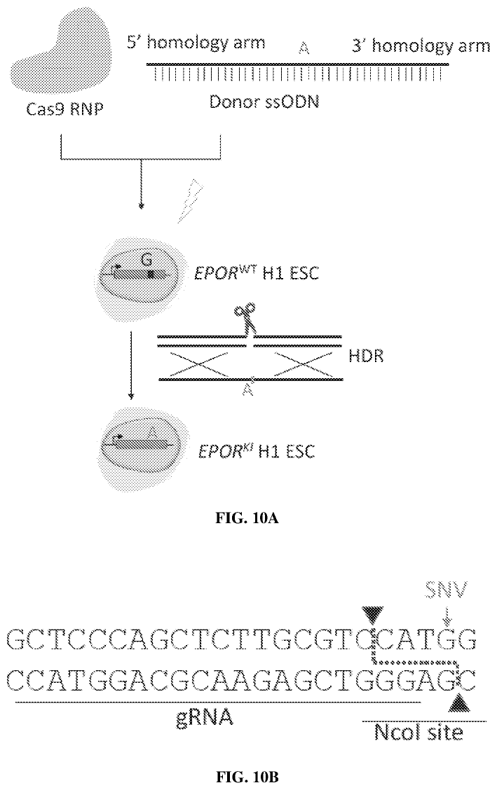

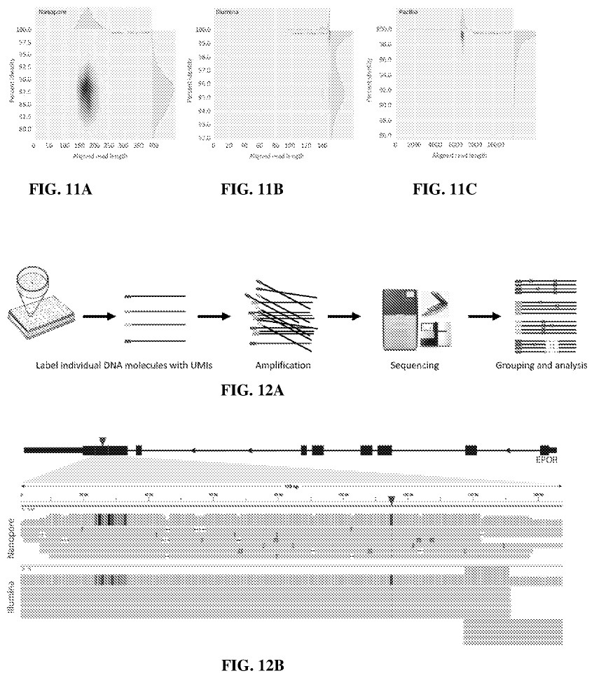

To determine if IDMseq can detect subclonal variants below the sensitivity limit of NGS (˜1% (Ley et al., Nature 456, 66-72 (2008), Zagordi et al., Nucleic Acids Res 38, 7400-7409 (2010))), synthetic cell populations harboring a mutation at various pre-determined allele frequencies were constructed. A homozygous single nucleotide variant (SNV) was knocked into the EPOR 20 gene using CRISPR-Cas9 in the H1 human embryonic stem cells (hESCs) (FIGS. 10A-10B). A rare subclonal mutation in a population of cells is simulated by admixing the genome of knock-in and wild-type cells at different ratios.

First, tests were executed to determine if IDMseq could overcome the high base-calling error of Nanopore sequencing in rare mutation detection. A 168 bp stretch of DNA encompassing the knock-in SNV was labeled with UMIs and amplified from a population with the ratio of 1:100 between knock-in and wild-type alleles.

A bioinformatic toolkit called Variant Analysis with UMI for Long-read Technology (VAULT) was also developed to analyze the sequencing data (see methods). The results showed that 36.5% of reads contained high-confidence UMI sequences (Table 4).

TABLE 4
Summary of Individual Sequencing Runs

| | | | | | | | | UMI | UMI | | | Somatic | |
|---|---|---|---|---|---|---|---|---|---|---|---|---|---|
| | | | | | | | | groups for | groups | | | SNV | |
| | | | | | | | Reads | variant | with | | Somatic | load | |
| | Mutant allele | | Amplicon | | Sequencing | Read | with | calling | introduced | | SNV | Per | SV |
| Gene | frequency (%) | | size | | platform | count | UMI | (>=5 reads) | mutation | | count | megabase | groups |
| EPOR | 1:100 | (1%) | 168 | bp | Nanopore | 17,634 | 6,444 | 284 | 2 | (0.7%) | 0 | N/A | N/A |
| EPOR | 1:1,000 | (0.1%) | 6,789 | bp | PacBio | 227,206 | 136,399 | 3,184 | 4 | (0.126%) | 273 | 8.9 | 3 |
| EPOR | 1:10,000 | (0.01%) | 168 | bp | Nanopore | 1,093,683 | 494,009 | 15,598 | 1 | (0.006%) | 10 | 3.8 | N/A |
| EPOR | 1:10,000 | (0.01%) | 168 | bp | Illumina | 7,488,257 | 7,236,007 | 132,341 | 5 | (0.004%) | 85 | 3.9 | N/A |

| | | | | | | | | | | | |
|---|---|---|---|---|---|---|---|---|---|---|---|
| PANX1 | N/A | 7077 | bp | Nanopore | 2,761,805 | 613,147 | 3,566 | N/A | 293 | 11.6 | 200 (5.6%) |
| PANX1 | N/A | 6595 | bp | Nanopore | 3,078,165 | 1,042,582 | 8,870 | N/A | 843 | 14.4 | 232 (2/6%) |

Based on a pre-set threshold of a minimum of 5 reads per UMI group, those reads are binned into 284 UMI groups. It is worth noting that every UMI group represents an original allele in the genome of the initial population. VAULT analysis showed that 2 UMI groups contained the knock-in SNV (FIGS. 11A-11C). Furthermore, no spurious mutation was detected. Importantly, when the trimmed reads were pooled for variant analysis without considering UMIs, no variant could be detected 35 by the same algorithms, proving the superior sensitivity afforded by IDMseq. These results suggest that IDMseq on the single-molecule Nanopore sequencing platform is able to accurately call rare variants without false positives.

Detection of rare variants in clinical settings often demands sensitivities well below that of prevailing NGS platforms (ca. 10⁻²). For instance, early cancer detection using circulating tumor DNA is estimated to require a sensitivity of at least 1 in 10,000 (Aravanis et al., Cell 168, 571-574 (2017)). To simulate this scenario, the same 168 bp region was sequenced in a population with the ratio of 1:10,000 between knock-in and wild-type alleles. It is worth noting that the UMI-labeling reaction contained only around 5 copies of the knock-in allele.

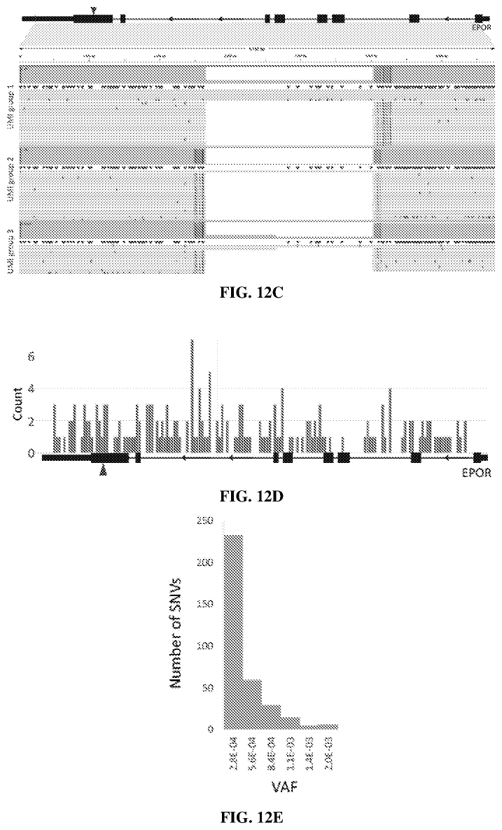

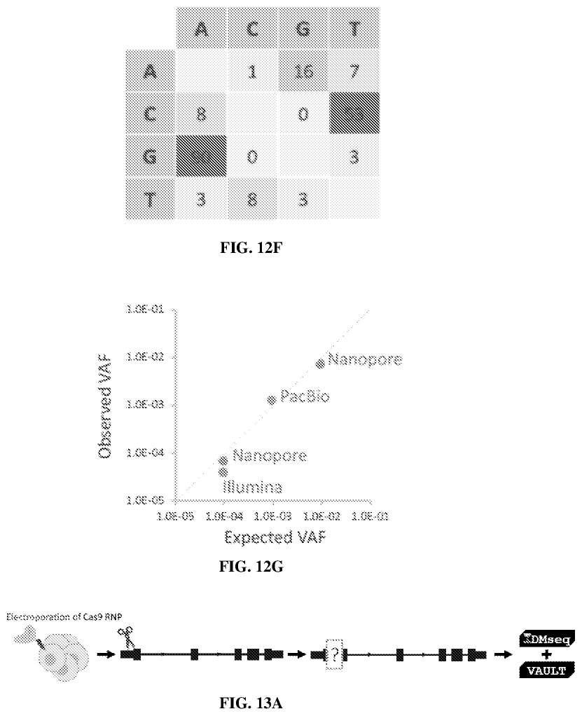

A 48-hour sequencing run on the MinION acquired 1.1 million reads (FIGS. 11A-11C). VAULT showed that 45.2% of reads contained high-confidence UMI sequences (Table 4). These reads were binned into 15,598 UMI groups of which one (0.6×10⁻⁴) contained the knock-in SNV (FIG. 12B). Ten other SNVs were also identified in ten UMI groups. Consideration was given as to whether or not there were PCR artifacts, as the main source of errors in UMI consensus sequencing originates from polymerase replication error in the barcoding step (Filges et al., Scientific reports 9, 3503 (2019)). The Platinum SuperFi DNA polymerase used has the highest reported fidelity (>300X that of Taq polymerase). It not only significantly reduces errors in the barcoding and amplification steps, but also captures twice more UMIs in the library than Taq (Filges et al., Scientific reports 9, 3503 (2019)). Theoretically, Platinum SuperFi polymerase introduces ˜6 errors in 10⁶ unique 168-bp molecules in the UMI-labeling step. Accordingly, this type of inescapable error is expected to be around 0.09 in 15,598 UMI groups, and thus cannot account for the observed SNV events. It was thus concluded that the ten SNVs are rare somatic mutations that reflect the genetic heterogeneity of hESCs as described previously (Merkle et al., Nature 545, 229-233 (2017)). These data provided an estimate of 3.8 somatic SNVs per megabase (Mb), which is consistent with the reported frequency of somatic mutation in coding sequence in normal healthy tissues (Martincorena et al., Science 348, 880-886 (2015)).

The length of 168-bp amplicon also allowed benchmarking against the industry standard Illumina sequencing, which features shorter reads but higher raw-read accuracy. The same 1:10,000 mixed population was then sequenced on an Illumina MiniSeq sequencer and obtained 7.5 million paired-end reads (FIG. 11A-11C). The results showed that 96.6% of reads contained high-confidence UMI sequences that were binned into 132,341 UMI groups, in which 5 (4×10⁻⁵) contained the knock-in SNV (Table 4, FIG. 12B). The calculated somatic SNV load was 3.9 per Mb, which closely matches the Nanopore data.

IDMseq was next applied to a larger region (6,789 bp) encompassing the knock-in SNV in a population with 0.1% mutant cells on a PacBio platform (FIGS. 11A-11C). VAULT showed that 60.0% of the high-fidelity long reads contain high-confidence UMIs, binned into 3,184 groups. Four UMI groups (1.26×10⁻³) contained only the knock-in SNV. Another 186 groups contained 273 SNVs (174 groups with 1 SNV, 9 groups with 2 SNVs, and 3 groups with 27 SNVs, Table 4). Again, 30 polymerase error during barcoding (˜0.82 error in 3,184 UMI groups) cannot account for the observed SNVs, indicating that most SNVs are true variants. Interestingly, structural variant (SV) analysis showed that the three groups with 27 SNVs shared the same 2,375 bp deletion. Haplotyping using the SNVs revealed that the three groups came from two haplotypes (FIG. 12C). This large deletion is far away from the Cas9 target site and thus less likely the result of genome editing. After excluding the SNVs in the large-deletion alleles, the remaining 192 SNVs distributed evenly in the region (FIG. 12D, Table 5).

TABLE 5
Summary of the frequency of SNVs in
different annotation categories.

| Type | Count | Percent |
|---|---|---|
| downstream | 150 | 27.1% |
| exon | 98 | 17.7% |
| intron | 263 | 47.6% |
| splice site region | 2 | 0.4% |
| transcript | 19 | 3.4% |
| UTR 3 prime | 21 | 3.8% |

Functional annotation of the SNVs showed that 17 of 192 caused an amino acid change. The spectrum of base changes and distribution of variant allele frequency (VAF) are consistent with published work (Martincorena et al., Science 348, 880-886 (2015)) (FIGS. 12E, 12F). These data provide an estimate of about 8.9 somatic SNVs per Mb.

Taken together these data showed that IDMseq provides reliable detection of rare variants (at least down to 10⁻⁴) and accurate estimate of variant frequency (FIG. 12G). It is useful for characterizing the spectrum of somatic mutations in human pluripotent stem cells (hPSCs).

Furthermore, it revealed a previously unappreciated phenomenon of spontaneous large deletion in hPSCs. Due to its large size and low frequency (VAF 0.1%), this SV would have been missed by short-read sequencing or ensemble long-read sequencing. Yet, it is conceivable that such an SV could confer growth advantage to the cell carrying it, and therefore has implications for the safety of hPSC in clinical settings. These findings nicely demonstrate the power of the combination of long-read sequencing and IDMseq in resolving complex genetic heterogeneity.

### Example

Despite its widespread adoption of the CRISPR-Cas9 system as an efficient and versatile genome-editing tool, the impact of CRIPSR on human genome integrity remains poorly understood (Kosicki et al., Nat Biotechnol 36, 765-771 (2018), Ihry et al., Nature medicine 24, 939-946 (2018), Haapaniemi et al., Nature medicine 24, 927-930 (2018)). Previous work indicated that the most prevalent DNA repair outcomes after Cas9 cutting are small indels (typically <20 bp) (van Overbeek, et al., Molecular Cell 63, 633-646 (2016), Koike-Yusa et al., Nature Biotechnology 32, 267-273 (2014)). Recent studies revealed large and complex SVs over several kilobases represent a significant portion of the on-target mutagenesis effect of Cas9 (Kosicki et al., Nat Biotechnol 36, 765-771 (2018), Adikusuma et al., Nature 560, E8-E9 (2018)). Importantly, to date, the analysis of large-deletion alleles came either from ensemble amplicon sequencing (Kosicki et al., Nat Biotechnol 36, 765-771 (2018), Adikusuma et al., Nature 560, E8-E9 (2018)) or whole-genome sequencing (Adikusuma et al., Nature 560, E8-E9 (2018)). The former is prone to amplification bias, and the latter cannot adequately detect large and complex variants due to the limited read length. Thus, IDMseq was applied to hESCs following CRISPR-Cas9 editing, to offer an unbiased quantification of the frequency and molecular feature of the DNA repair outcomes of double-strand breaks induced by Cas9.

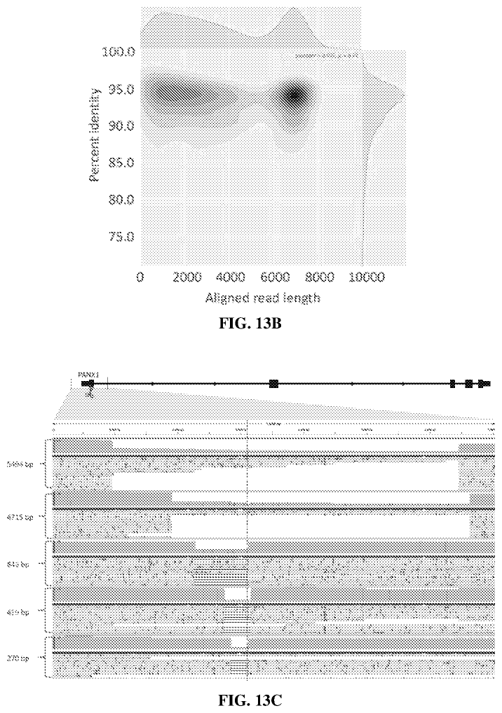

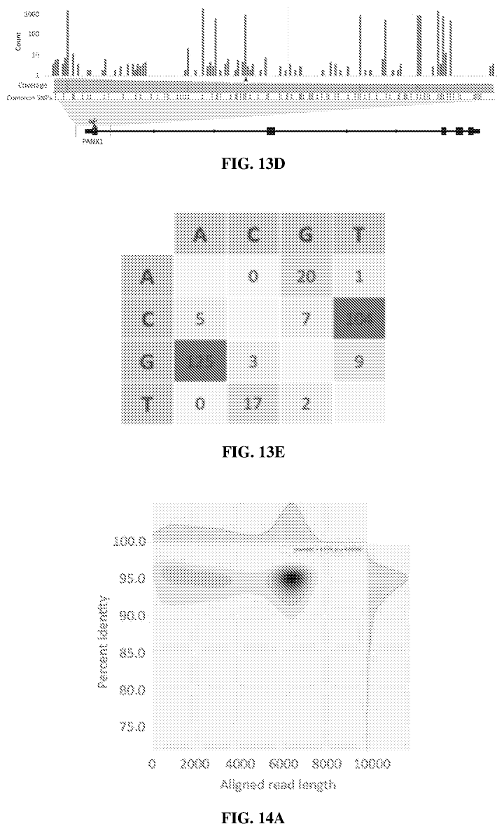

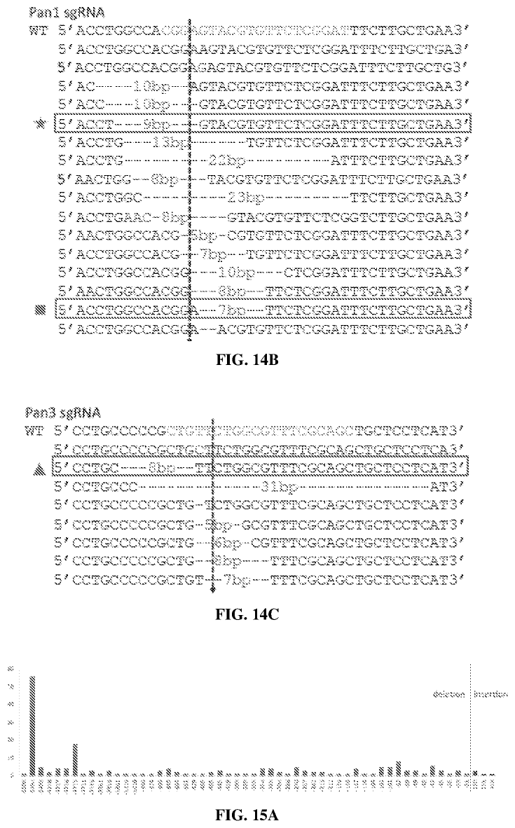

Exon 1 (Pani) and exon 3 (Pan3) of the Pannexin 1 (PANX1) gene were targeted with two efficient gRNAs (FIG. 13A). Forty-eight hours after electroporation of Cas9 complexed with the Pan1 or Pan3 gRNA, H1 ESCs were harvested for IDMseq. The surveyed region is 7,077 bp for Pan1 and 6,595 bp for Pan3. A 48 h Nanopore sequencing run yielded 2.8 million and 3.1 million reads for Pan1 and Pan3, which were binned into 3,566 and 8,870 UMI groups, respectively (Table 4, FIG. 13B, FIG. 14A).

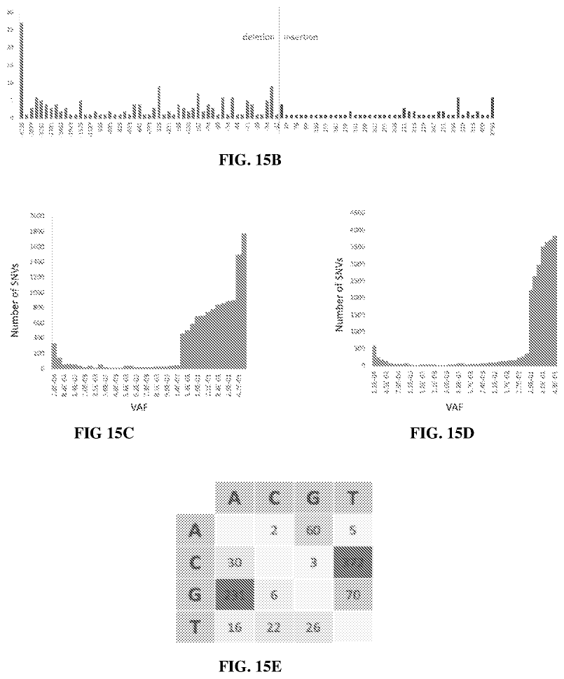

First, SVs (>30 bp) were surveyed in UMI groups. After SV calling and filtering out lowly supported SVs (see methods), 200 (5.6%) of the 3,566 UMI groups contained 200 SVs in Pan1-edited cells, including 195 deletions and 5 insertions. The size of SVs ranged from 31 to 5,506 bp (FIG. 13C, FIG. 15A). Intriguingly, some large deletions were independently captured multiple times. For 30 example, 56 (28.0%) UMI groups have the same 5,494-bp deletion and 18 (9.0%) UMI groups have the same 4,715-bp deletion. For the insertion variants, 3 of the 5 UMI groups shared the same SV.

When a different gRNA (Pan3) was used, 232 (2.6%) of 8,870 UMI groups contained 240 SVs 35 (178 deletions, 50 insertions and 12 inversions), with size ranging from 31 to 4,238 bp (FIG. 15B). Importantly, reoccurring SVs were also detected with Pan3. Twenty-seven (32.1%) UMI groups shared the same 4,238-bp deletion, and 6 (2.5%) groups shared a 2,750-bp insertion. These data provided the first quantitative evidence that the repair outcome of Cas9 cutting is not random and there are likely hotspots for Cas9-induced large deletions or insertions.

Next SNVs were analyzed in these two data sets. Cas9 editing with the Pan1- and Pan3 gRNAs resulted in similar SNV patterns (FIG. 13D). The results of Pan1 showed that 2,731 (76.6%) of 3,566 UMI groups contained 10,871 SNPs, while for Pan3 8,018 (90%) of 8,870 UMI groups contained 23,477 SNVs. In both cases, the SNVs fell into two frequency ranges. Most SNVs in the high-frequency category (FIG. 13D) have been reported in the common SNP database. The low-frequency SNVs (FIG. 13D) distributed evenly in the locus and did not overlap with known SNPs, likely representing somatic mutations. There was no clear enrichment of SNVs around the cutting site, which is consistent with previous reports (Wang et al., BMC Genomics 19, 397 (2018)). The frequency of presumed somatic mutations for Pan1 (293 somatic mutations) and Pan3 (843 somatic mutations) is 11.6 and 14.4 per Mb, respectively, which is slightly higher than the average value (˜7/Mb) reported by NGS (Martincorena et al., Science (New York, N.Y 348, 880-886 (2015)). The spectrum (FIGS. 13D-13E, Table 6) and VAF (FIGS. 15C-15E, Table 7) of single nucleotide substitutions were consistent with published data (Martincorena et al., Science (New York, N.Y 348, 880-886 (2015)).

TABLE 6
Analysis of somatic mutations detected in CRISPR-
edited hESCs based on functional annotation.

| Type | Count | Percent |
|---|---|---|
| exon | 20 | 4.5% |
| intergenic | 139 | 31.5% |
| intron | 107 | 24.3% |
| transcript | 7 | 1.6% |
| upstream | 139 | 31.5% |
| UTR 5 prime | 29 | 6.6% |

TABLE 7
Analysis of somatic mutations detected in Pan3-
edited hESCs based on functional annotation.

| Type | Count | Percent |
|---|---|---|
| exon | 149 | 16.2% |
| intron | 639 | 69.3% |
| splice site acceptor | 3 | 0.3% |
| transcript | 79 | 8.6% |
| UTR 3 prime | 52 | 5.6% |

Besides SNVs and SVs, VAULT also reported many small indels around the Cas9 cleavage site. The indels were compared with the Sanger sequencing data of single-cell derived clones. The results showed that IDMseq correctly identified a subset of the deletion alleles (FIGS. 14B-14C).

In summary, IDMseq and VAULT enable quantitation and haplotyping of both small and large genetic variants at the subclonal level. They are easy to implement and compatible with all current sequencing platforms, including the portable Oxford Nanopore MinION. IDMseq provides an unbiased base-resolution characterization of on-target mutagenesis induced by CRISPR-Cas9, which could facilitate the safe use of the CRISPR technology in the clinic. The high sensitivity afforded by IDMseq and VAULT may be useful for early cancer detection using circulating tumor DNA or detection of minimal residual diseases. Results showed that IDMseq is accurate in profiling rare somatic mutations, which could aid the study of genetic heterogeneity in tumors or aging tissues. IDMseq in its current form only sequences one strand of the DNA duplex, and its performance may be further improved by sequencing both strands of the duplex.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A unique molecular identifier (UMI) primer comprising a universal primer sequence, a unique molecular identifier (UMI) sequence, and a first target nucleic acid binding sequence.

2. The primer of claim 1 wherein the orientation of the universal primer sequence, unique molecular identifier (UMI) sequence, and first target nucleic acid binding sequence is 5′ universal primer sequence, unique molecular identifier (UMI) sequence, first target nucleic acid binding sequence 3′.

3. The primer of claim 1 wherein: (a) the universal primer sequence comprises the sequence CATCTTACGATTACGCCAACCAC (SEQ ID NO:1), the reverse sequence thereof, the complementary sequence thereto, the reverse complementary sequence thereof.; (b) the UMI sequence comprises a random sequence (such as NNNN or NNNNNNN), a partially degenerate nucleotide sequence (such as NNNRNYN or NNNNTGNNNN (SEQ ID NO:2), wherein “N” can be A, T, G, or C, “R” can be G or A, and “Y” can be T or C, or the reverse sequence thereof, the complementary sequence thereto, or the reverse complementary sequence thereof, optionally wherein the UMI sequence is between about 5 and about 100 nucleotides in length; and/or the first target nucleic acid binding sequence binds at or near or a gene of interest; optionally, wherein the first target nucleic acid binding sequence binds to mitochondrial DNA.

4. (canceled)

5. (canceled)

6. (canceled)

7. The primer of claim 1 comprising CATCTTACGATTACGCCAACCACTGNNNTGNNNCTCCCGAATCAACCCTGACCC (SEQ ID NO:3)

8. A method of labeling a target nucleic acid comprising carrying out at least one cycle of polymerase chain reaction using a first primer of claim 1 and a nucleic acid sample comprising a nucleic acid sequence to which the first target nucleic acid binding sequence of the primer can bind.

9. The method of claim 8 wherein: (a) the first cycle of PCR further comprises a second primer comprising a second target nucleic acid binding sequence and the target nucleic acid comprises a nucleic acid sequence to which the second target nucleic acid binding sequence of the second primer can bind; and/or (b) a second and optionally one or more subsequent cycles of PCR further comprises a second primer alone or in combination with the first primer, the second primer comprising a second target nucleic acid binding sequence, and the target nucleic acid comprising a nucleic acid sequence to which the second target nucleic acid binding sequence of the second primer can bind; and/or
(i) the second primer further comprises the same or a different universal primer sequence as the first primer, or the reverse sequence thereof, the complementary sequence thereto, or the reverse complementary sequence thereof;
(ii) the second primer further comprises the same or different UMI as the first primer, or the reverse sequence thereof, the complementary sequence thereto, or the reverse complementary sequence thereof; or
(ii) the orientation of the universal primer sequence, unique molecular identifier (UMI) sequence, and second target nucleic acid binding sequence of the second primer is 5′ universal primer sequence, unique molecular identifier (UMI) sequence, second target nucleic acid binding sequence 3′

10. (canceled)

11. (canceled)

12. (canceled)

13. (canceled)

14. The method of claim 9 comprising any integer between 1 and 100 inclusive subsequent cycles of PCR.

15. The method of claim 8, wherein:
(a) the nucleic acid sample is nuclear genomic DNA, mitochondrial genomic DNA, or a combination thereof:,
(b) the source of the nucleic acid sample is any integer between 1 and 1,000,000 cells inclusive, or any range formed of two integers there between, for example, between 1 and 10,000, 1 and 1,000, 1 and 100, 1 and 10, or 1 single cell;
(c) the source of the nucleic acid sample is one single nuclei or one single mitochondrion;
(d) the nucleic acid sample is isolated from a cell or cells;
(e) the isolation comprises releasing the target nucleic acid sample by lysing the cell(s); and/or
(f) the nucleic acid sample is subjected to a restriction digestion prior to the first cycle of PCR.

16. (canceled)

17. (canceled)

18. (canceled)

19. (canceled)

20. (canceled)

21. The method claim 8 further comprising: (a) removing contaminants (e.g., one or more of primers, dNTPs, RNA, etc.), before the first cycle of PCR, after the first cycle of PCR, after the second cycle of PCR, after the last cycle of PCR, or any combination thereof; (b) any integer between 1 and 100 inclusive cycles of PCR comprising primers that bind to the one or more universal primer sequences alone or in combination with a target nucleic acid specific primer, wherein the cycles of PCR amplify one- or two-end UMI labeled target nucleic acid; (c) amplifying the nucleic acid sample, or a fraction thereof, prior to labeling; and/or (d) one or more rounds of enrichment and/or purification of the nucleic acid sample, target nucleic acid, amplicons, or otherwise labeled nucleic acid; and/or wherein the enrichment and/or purification comprises size selection.

22. (canceled)

23. The method of claim 8 comprising two or more first and second primer sets, each first and second primer set comprising different target nucleic acid binding sequences designed to label and optionally amplify different target nucleic acids.

24. The method of claim 23, wherein:
(a) the UMI sequence for each first primer of each primer set is the same;
(b) the UMI sequence for each first primer of each primer set is different;
(c) the UMI sequence for each second primer of each primer set is the same; and/or
(d) the UMI sequence for each second primer of each primer set is different

25. (canceled)

26. (canceled)

27. (canceled)

28. A method of determining the sequence of a target nucleic acid comprising
(i) labeling one or more target nucleic acids according to the method of any one of claim 8;
(ii) sequencing the labeled amplicons;
(iii) optionally grouping sequences having the same UMI into one of more groups;
(iv) determining the sequence of each target nucleic acid sequence by determining the consensus sequence of each group.

29. The method of claim 28 further comprising (v) identifying polymorphisms in one or more of the target nucleic acids, optionally, wherein the polymorphism is a single nucleotide polymorphism (SNP).

30. (canceled)

31. The method of claim 28, wherein the sequence comprises long-read sequencing technology and optionally, wherein the long-read sequencing technology is comprises Nanopore MinION sequencer, or the long-read sequencing technology comprises preparing a 1D ligation library from the labeled amplicons.

32. (canceled)

33. (canceled)

34. The method of claim 28, wherein any of steps (iii)-(v) are carried out using bioinformatics analysis, wherein the bioinformatics analysis comprises basecalling, sequence alignment(s), polymorphism identification or a combination thereof; or the bioinformatics analysis comprises one or more of steps of FIG. 3C.

35. (canceled)

36. (canceled)

37. (canceled)

38. A method of labeling a target nucleic acid and optionally sequencing the labeled target nucleic comprising
(i) restriction enzyme (e.g., BsrG1) digest of only the nuclear DNA in a nucleic acid sample comprising nuclear and mitochondrial DNA;
(ii) treatment of the nucleic acid sample with lambda exonuclease;
(iii) labeling of the remaining mtDNA with UMI labels, priming sites, and bar codes using EZ-Tn5 transposon;
(iv) sequencing the labeled mtDNA.

39. (canceled)

40. (canceled)

41. (canceled)

42. The method of claim 8, wherein the target nucleic acid is, or is suspected of, being related to aging or an age-related disorder.

43. The method of claim 8, comprising: (a) one-end UMI labeling comprising a single round of extension of a UMI primer comprising a universal primer sequence, unique molecular identifier sequence, and target nucleic acid binding sequence that hybridizes to a target nucleic acid sequence and optionally removing the UMI primer from the reaction mixture; or (b) two-end UMI labeling comprising a single round of extension of a forward UMI primer comprising a universal primer sequence, unique molecular identifier sequence, and target nucleic acid binding sequence that hybridizes to a target nucleic acid sequence and optionally removing the forward UMI primer from the reaction mixture, and a single round of extension of a reverse UMI primer comprising a universal primer sequence, unique molecular identifier sequence, and target nucleic acid binding sequence that hybridizes to a target nucleic acid sequence and optionally removing the reverse UMI primer from the reaction mixture.)

44. (canceled)

45. The methods of claim 43, further comprising amplifying the one-end or two-end labeled target nucleic acids by PCR with a universal primer alone or in combination with a target nucleic acid specific primer, wherein the cycles of PCR amplify the one- or two-end UMI labeled target nucleic acid.

46. A method of determining the sequence of a target nucleic acid comprising
(i) labeling one or more target nucleic acids according to the method of claim 43;
(ii) sequencing the labeled amplicons;
(iii) optionally grouping sequences having the same UMI into one of more groups;
(iv) determining the sequence of each target nucleic acid sequence by determining the consensus sequence of each group.

47. (canceled)
